(19) United States
(12) Patent Application Publication
(10) Pub. No.: US 2023/0120086 A1
(43) Pub. Date: Apr. 20, 2023

(54) SURGICAL EXTRACTOR

(71) Applicant: Shukla Medical, St. Petersburg, FL (US)

(72) Inventor: Adam Gosik-Wolfe, Tampa, FL (US)

(73) Assignee: Shukla Medical, St. Petersburg, FL (US)

(21) Appl. No.: 18/057,253

(22) Filed: Nov. 21, 2022

Related U.S. Application Data
(63) Continuation-in-part of application No. 17/204,385, filed on Mar. 17, 2021.
(60) Provisional application No. 62/991,940, filed on Mar. 19, 2020.

(51) Int. Cl. A61B 17/28

(52) U.S. Cl. CPC A61B 17/282

(57) ABSTRACT

A surgical extractor including a second arm having a proximal end and a distal end, and a first arm having a proximal end for attachment to an extraction device. The first arm additionally has a distal end for attachment to a first modular jaw. The first arm further includes a transverse opening having fastener structure configured to releasably retain an extraction impactor or an extraction device connector. The surgical extractor additionally includes a link pivotably connected to the first and second arms, the link having a distal end for attachment to a second modular jaw.

### CROSS-REFERENCE TO RELATED APPLICATIONS

This application is continuation-in-part of U.S. patent application Ser. No. 17/204,385, filed Mar. 17, 2021, and entitled “Implant Extractor,” which claims the benefit under 35 U.S.C. § 119(e) to U.S. Provisional Patent Application No. 62/991,940, filed Mar. 19, 2020, and entitled “Glenosphere Extractor,” the entire disclosures of which are hereby incorporated by reference for all purposes.

### BACKGROUND OF THE DISCLOSURE

Surgical extractor tools are typically either simple mechanical devices that do not effectively grip an object to be extracted from the body or complex mechanical devices that are expensive to manufacture and difficult to operate.

### BRIEF SUMMARY OF THE DISCLOSURE

The exemplary embodiments of present invention relate generally to a surgical extractor tool and, more specifically, to a tool for extracting objects such as implants and surgical hardware from the body including, without limitation, intramedullary nails, rods and wires, bone screws, surgical hex nuts, revision sleeves, femoral head implants, glenosphere implants and other implants.

In accordance with an exemplary embodiment there is provided a surgical extractor comprising a second arm having a proximal end and a distal end, and a first arm having a proximal end for attachment to an extraction device. The first arm additionally has a distal end for attachment to a first jaw. The surgical extractor additionally comprises a link pivotably connected to the first and second arms, the link having a distal end for attachment to a second jaw. The surgical extractor further comprises a first jaw releasably attachable to the distal end of the first arm and a second jaw releasably attachable to the distal end of the link.

According to aspect, the first and second jaws each include a slidable lock to slidingly engage a corresponding slidable lock on the first arm and link, respectively. According to another aspect, the corresponding slidable lock on the first arm and link each includes a stop. According to another aspect, the stop on the first arm is a laterally extending stop and the stop of the link is a laterally extending stop. According to another aspect, the slidable lock is a dovetail. According to another aspect, the slidable lock on each of the first and second jaws is a male dovetail and the corresponding slidable lock on each of the first arm and the link is a female dovetail. According to another aspect, the surgical extractor further comprises a detent carried by one of the first jaw and the first arm, or a detent carried by one of the second jaw and the link.

In accordance with another exemplary embodiment there is provided a surgical extractor comprising a second arm having a proximal end and a distal end, and a first arm having a proximal end for attachment to an extraction device. The first arm additionally has a distal end for attachment to a first jaw, and a fastener configured to releasably retain an extraction impactor or an extraction device connector. The surgical extractor additionally comprises a link pivotably connected to the first and second arms, the link having a distal end for attachment to a second jaw.

According to an aspect, the surgical extractor further comprises an extraction impactor or an extraction device connector having a cooperating fastener for engaging the fastener of the first arm. According to another aspect, the fastener and the cooperating fastener are cooperating threading, a J-slot connection, a press-fit, a Luer lock, a snap fit, or a ball detent.

According to an aspect, the extraction impactor comprises a radially projecting knob defining a striking surface. According to another aspect, the radially projecting knob comprises a plurality of grip enhancing notches provided on a periphery thereof.

According to an aspect, the extraction device connector comprises a base for receiving a strike plate. According to another aspect, the base is polygonal in shape. According to another aspect, the extraction device connector comprises a quick connect configured to releasably engage with an extraction device.

According to an aspect, the extraction impactor or the extraction device connector extends laterally away from the first arm when connected to the fastener. According to another aspect, the fastener is an opening transverse to a longitudinal axis of the first arm. According to another aspect, the fastener is spaced the distal end of the first arm. According to another aspect, the fastener is positioned about a midportion of the first arm. According to another aspect, the fastener is between the distal end of the first arm for attaching to the first jaw and a proximal end of the first arm.

According to an aspect, a gripping face of a bite portion of at least one of the first jaw and the second jaw comprises a plurality of transverse teeth and a longitudinal groove separating the plurality of transverse teeth, a projection which stands proud of the gripping face and is configured to engage a slot of an object, a lip-like projection which stands proud of the gripping face and is configured to engage a distal face of an object, a plurality of transverse teeth arranged in a concave arc, or a plurality of transversely extending teeth and a plurality of longitudinally extending teeth.

According to an aspect, a bite portion of the first jaw comprises a pair of elongated bifurcated arms each having a downwardly depending lug at a distal end thereof, wherein a bite portion of the second jaw comprises a single elongated arm having an upwardly projecting lug at a distal end thereof, and wherein the single elongated arm of the second jaw is operable to pass through a gap defined between the pair of elongated bifurcated arms of the first jaw, whereby the lugs of the elongated bifurcated arms and the lug of the single elongated arm engage a perimeter of an opening of an object.

According to an aspect, a bite portion of the first jaw comprises a pair of sloped surfaces defining an acute angle for engaging first and second spaced apart surfaces of a hex nut, and wherein a bite portion of the second jaw has a convex surface for engaging a third surface of the hex nut that is spaced apart from the first and second spaced apart surfaces of the hex nut.

According to an aspect, a bite portion of the first jaw comprises a pair of downwardly depending arms at a distal end thereof, and wherein a bite portion of the second jaw curves downwardly from a proximal end to a distal end thereof.

Other features and advantages of the subject disclosure will be apparent from the following more detailed description of the exemplary embodiments.

### BRIEF DESCRIPTION OF THE SEVERAL VIEWS OF THE DRAWINGS

The foregoing summary, as well as the following detailed description of the exemplary embodiments of the subject disclosure, will be better understood when read in conjunction with the appended drawings. For the purpose of illustrating the present disclosure, there are shown in the drawings exemplary embodiments. It should be understood, however, that the subject application is not limited to the precise arrangements and instrumentalities shown.

FIG. 1 is side view of a surgical extractor in accordance with an exemplary embodiment of the subject disclosure;

FIG. 2 is a posterior side view of the surgical extractor of FIG. 1;

FIG. 3 is an exploded side view of the surgical extractor of FIG. 1 with certain elements shown in phantom line for purposes of clarity;

FIG. 4A is an exploded perspective view of an extraction device, strike plate and quick connect of the surgical extractor of FIG. 1;

FIG. 4B is a plan view of a strike plate of the surgical extractor of FIG. 1;

FIG. 5 is a longitudinal cross-sectional side view of the surgical extractor of FIG. 1;

FIG. 6 is a rear perspective view of the surgical extractor of FIG. 1 with a rotatable knob shown separated from the surgical extractor;

FIG. 7A is side view of a second arm of the surgical extractor of FIG. 1;

FIG. 7B is a perspective view of the second arm of FIG. 7A;

FIG. 8A is a rear perspective view of a first arm of the surgical extractor of FIG. 1;

FIG. 8B is a front perspective view of the first arm of FIG. 8A;

FIG. 8C is a front view of the first arm of FIG. 8A;

FIG. 8D is a cross-sectional side view of the first arm of FIG. 8A;

FIG. 8E is a side view of the first arm of FIG. 8A including a threaded rod threadedly engaging the first arm;

FIG. 9A is a perspective view of a threaded rod of an adjuster of the surgical extractor of FIG. 1;

FIG. 9B is an end view of the threaded rod of FIG. 9A;

FIG. 10A is a side view of a rotatable knob of an adjuster of the surgical extractor of FIG. 1;

FIG. 10B is an end view of the rotatable knob of FIG. 10A;

FIG. 11 is a left perspective view of a lever of the surgical extractor of FIG. 1;

FIGS. 12A-12C are various views of a link of the surgical extractor of FIG. 1 in accordance with an exemplary embodiment of the subject disclosure;

FIG. 13 is a rear perspective of the surgical extractor of FIG. 1 with first and second jaws thereof omitted for purposes of clarity;

FIG. 14 is a rear perspective view of a first jaw of the surgical extractor of FIG. 1,

FIG. 15 is a rear perspective view of a second jaw of the surgical extractor of FIG. 1;

FIG. 16 is a perspective view of the lever, the second arm and the locking mechanism of the surgical extractor of FIG. 1 in an unlocked position;

FIG. 17 is a top view of the lever, the second arm and the locking mechanism of FIG. 16 in an unlocked position;

FIG. 18 is a top view of the lever, the second arm and the locking mechanism of FIG. 16 in a locked position;

FIG. 19 is a perspective view of a release lever and the second arm of the implant extractor of FIG. 1 with the second arm shown in phantom line for purposes of clarity;

FIG. 20A is a side view of an alternative configuration of the first jaw of the surgical extractor of FIG. 1;

FIG. 20B is a perspective view of the alternative configuration of the first jaw of FIG. 20A;

FIG. 20C is a perspective view of the alternative configuration of the first jaw of FIG. 20A;

FIG. 21A is a side view of an alternative configuration of the second jaw of the implant extractor of FIG. 1;

FIG. 21B is a perspective view of the alternative configuration of the second jaw of FIG. 21A;

FIG. 21C is a perspective view of the alternative configuration of the second jaw of FIG. 21A;

FIG. 22A is a bottom perspective view of an another exemplary embodiment of the surgical extractor in accordance with the subject disclosure;

FIG. 22B is a top perspective view of the surgical extractor of FIG. 22A;

FIG. 22C is an side view of the surgical extractor of FIG. 22A;

FIG. 23 is an elevation view of a connector configured for use in the surgical extractor of FIGS. 22A-22C;

FIG. 24A is a perspective view of yet another exemplary embodiment of a surgical extractor in accordance with the subject disclosure;

FIG. 24B is side view of the surgical extractor of FIG. 24A;

FIG. 25A is a top perspective view of an extraction impactor of the surgical extractor of FIGS. 24A and 24B;

FIG. 25B is a side view of the extraction impactor of FIG. 25A;

FIG. 26A is a side perspective view of alternative configurations of the first and second jaws of the surgical extractor of FIG. 1 shown gripping an object;

FIG. 26B is a bottom perspective view of the alternative configuration of the first jaw of FIG. 26A;

FIG. 26C is a top perspective view of the alternative configuration of the second jaw of FIG. 26A;

FIG. 27A is a side perspective view of alternative configurations of the first and second jaws of the surgical extractor of FIG. 1 shown gripping an object;

FIG. 27B is a bottom perspective view of the alternative configuration of the first jaw of FIG. 27A;

FIG. 27C is a top perspective view of the alternative configuration of the second jaw of FIG. 27A;

FIG. 28A is a side perspective view of alternative configurations of the first and second jaws of the surgical extractor of FIG. 1 shown gripping an object;

FIG. 28B is a bottom perspective view of the alternative configuration of the first jaw of FIG. 28A;

FIG. 28C is a top perspective view of the alternative configuration of the second jaw of FIG. 28A;

FIG. 29A is a side perspective view of alternative configurations of the first and second jaws of the surgical extractor of FIG. 1 shown gripping an object;

FIG. 29B is a bottom perspective view of the alternative configuration of the first jaw of FIG. 29A;

FIG. 29C is a top perspective view of the alternative configuration of the second jaw of FIG. 29A;

FIG. 30A is a side perspective view of alternative configurations of the first and second jaws of the surgical extractor of FIG. 1 shown gripping an object;

FIG. 30B is a bottom perspective view of the alternative configuration of the first jaw of FIG. 30A;

FIG. 30C is a top perspective view of the alternative configuration of the second jaw of FIG. 30A;

FIG. 31A is a side perspective view of alternative configurations of the first and second jaws of the surgical extractor of FIG. 1 shown gripping an object;

FIG. 31B is a bottom perspective view of the alternative configuration of the first jaw of FIG. 31A;

FIG. 31C is a top perspective view of the alternative configuration of the second jaw of FIG. 31A;

FIG. 32A is a side perspective view of alternative configurations of the first and second jaws of the surgical extractor of FIG. 1 shown gripping an object;

FIG. 32B is a bottom perspective view of the alternative configuration of the first jaw of FIG. 32A;

FIG. 32C is a top perspective view of the alternative configuration of the second jaw of FIG. 32A;

FIG. 33A is a side perspective view of alternative configurations of the first and second jaws of the surgical extractor of FIG. 1 shown gripping an object;

FIG. 33B is a bottom perspective view of the alternative configuration of the first jaw of FIG. 33A;

FIG. 33C is a top perspective view of the alternative configuration of the second jaw of FIG. 33A;

FIG. 34A is a perspective view of the surgical extractor of FIG. 1 equipped with alternative configurations of the first and second jaws shown engaging an object;

FIG. 34B is a top perspective view of the alternative configuration of the first jaw shown in FIG. 34A;

FIG. 34C is a side view of the alternative configuration of the first jaw shown in FIG. 34A;

FIG. 34D is a top plan view of the alternative configuration of the first jaw shown in FIG. 34A;

FIG. 34E is a top perspective view of the alternative configuration of the second jaw shown in FIG. 34A;

FIG. 34F is a side view of the alternative configuration of the second jaw shown in FIG. 34A;

FIG. 34G is a top plan view of the alternative configuration of the second jaw shown in FIG. 34A;

FIG. 34H is a perspective view showing the interaction of the first and second jaws of FIG. 34A;

FIG. 35A is a side perspective view of alternative configurations of the first and second jaws of the surgical extractor of FIG. 1 shown gripping an object;

FIG. 35B is a bottom perspective view of the alternative configuration of the first jaw shown in FIG. 35A;

FIG. 35C is a side view of the alternative configuration of the first jaw shown in FIG. 35A;

FIG. 35D is a front view of the alternative configuration of the first jaw shown in FIG. 35A;

FIG. 35E is a rear view of the alternative configuration of the first jaw shown in FIG. 35A;

FIG. 35F is a top perspective view of the alternative configuration of the second jaw shown in FIG. 35A;

FIG. 35G is a side view of the alternative configuration of the second jaw shown in FIG. 35A;

FIG. 35H is a front view of the alternative configuration of the second jaw shown in FIG. 35A;

FIG. 35I is a rear view of the alternative configuration of the second jaw shown in FIG. 35A;

FIG. 36A is a front perspective view of alternative configurations of the first and second jaws of the surgical extractor of FIG. 1 shown gripping an object;

FIG. 36B is a side view of the alternative configurations of the first and second jaws of FIG. 36A shown gripping an object;

FIG. 36C is a bottom perspective view of the alternative configuration of the first jaw shown in FIG. 36A;

FIG. 36D is a side view of the alternative configuration of the first jaw shown in FIG. 36A;

FIG. 36E is a front view of the alternative configuration of the first jaw shown in FIG. 36A;

FIG. 36F is a rear view of the alternative configuration of the first jaw shown in FIG. 36A;

FIG. 36G is a top perspective view of the alternative configuration of the second jaw shown in FIG. 36A;

FIG. 36H is a side view of the alternative configuration of the second jaw shown in FIG. 36A;

FIG. 36I is a front view of the alternative configuration of the second jaw shown in FIG. 36A;

FIG. 36J is a rear view of the alternative configuration of the second jaw shown in FIG. 36A;

FIG. 37A is a bottom perspective view of an alternative configuration of a first jaw of the surgical extractor of FIG. 1; and

FIG. 37B is a bottom perspective view of an alternative configuration of a second jaw of the surgical extractor of FIG. 1.

### DETAILED DESCRIPTION OF THE DISCLOSURE

Reference will now be made in detail to the various exemplary embodiments of the subject disclosure illustrated in the accompanying drawings. Wherever possible, the same or like reference numbers will be used throughout the drawings to refer to the same or like features. It should be noted that the drawings are in simplified form and are not drawn to precise scale. Certain terminology is used in the following description for convenience only and is not limiting. Directional terms such as top, bottom, left, right, above, below and diagonal, are used with respect to the accompanying drawings. The term “distal” shall mean away from the center of a body. The term “proximal” shall mean closer towards the center of a body and/or away from the “distal” end. The words “inwardly” and “outwardly” refer to directions toward and away from, respectively, the geometric center of the identified element and designated parts thereof. Such directional terms used in conjunction with the following description of the drawings should not be construed to limit the scope of the subject application in any manner not explicitly set forth. Additionally, the term “a,” as used in the specification, means “at least one.” The terminology includes the words above specifically mentioned, derivatives thereof, and words of similar import.

“About” as used herein when referring to a measurable value such as an amount, a temporal duration, and the like, is meant to encompass variations of ±20%, ±10%, ±5%, ±1%, or ±0.1% from the specified value, as such variations are appropriate.

“Substantially” as used herein shall mean considerable in extent, largely but not wholly that which is specified, or an appropriate variation therefrom as is acceptable within the field of art. “Exemplary” as used herein shall mean serving as an example.

Throughout the subject application, various aspects thereof can be presented in a range format. It should be understood that the description in range format is merely for convenience and brevity and should not be construed as an inflexible limitation on the scope of the subject disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Furthermore, the described features, advantages and characteristics of the exemplary embodiments of the subject disclosure may be combined in any suitable manner in one or more embodiments. One skilled in the relevant art will recognize, in light of the description herein, that the subject disclosure can be practiced without one or more of the specific features or advantages of a particular exemplary embodiment.

In other instances, additional features and advantages may be recognized in certain embodiments that may not be present in all exemplary embodiments of the present disclosure.

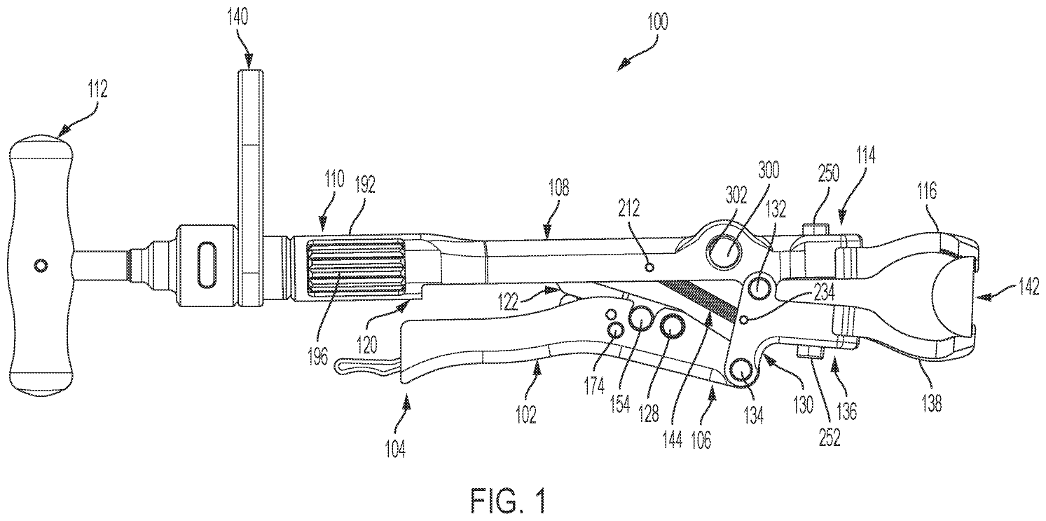

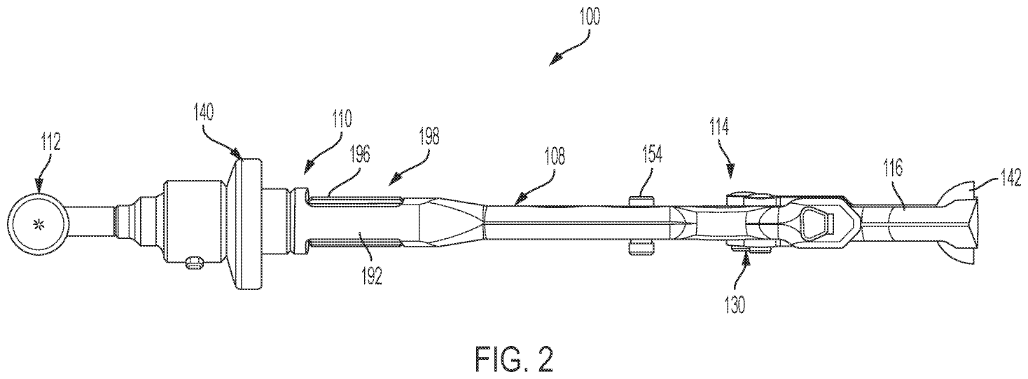

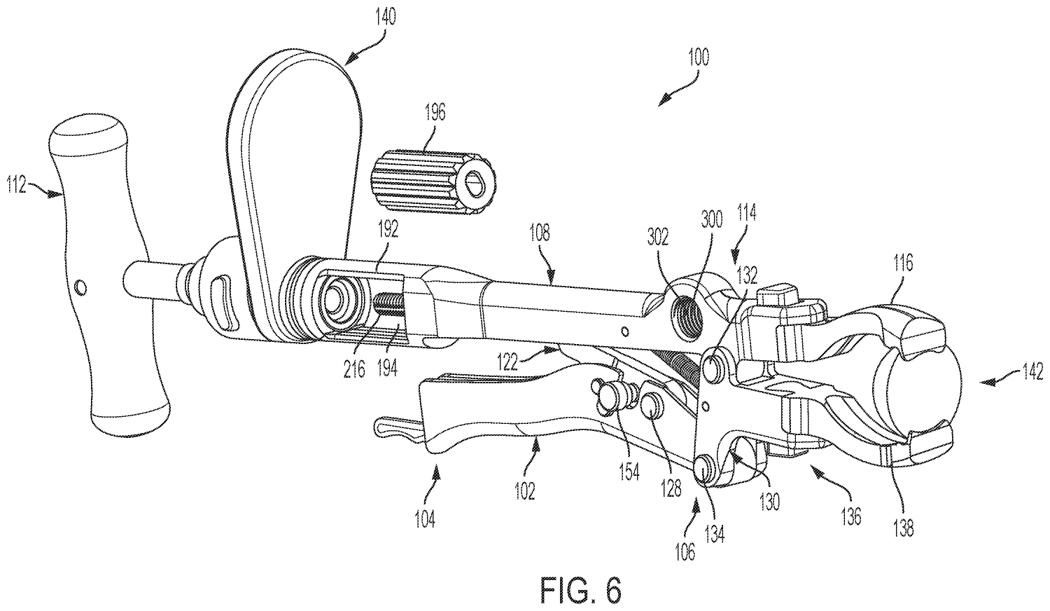

Referring now to the drawings, FIGS. 1-3, 5 and 6 illustrate a surgical extractor 100 in accordance with an exemplary embodiment of the present disclosure. The surgical extractor 100 includes a second arm 102 having a proximal end 104 and a distal end 106, and a first arm 108 having a proximal end 110 for attachment to an extraction device 112. The extraction device 112 can be any suitable extraction device including, without limitation, a T-handle. The first arm additionally has a distal end 114 for attachment to a first modular jaw 116, and an adjustment mechanism 118 including an adjuster 120 and a lever 122. The lever has a proximal end 124 engaged with the adjuster and a distal end 126 pivotably connected to the second arm 102 via pivot pin 128. The surgical extractor 100 additionally comprises a link 130 pivotably connected to the first and second arms via pivot pins 132, 134, the link having a distal end 136 for attachment to a second modular jaw 138.

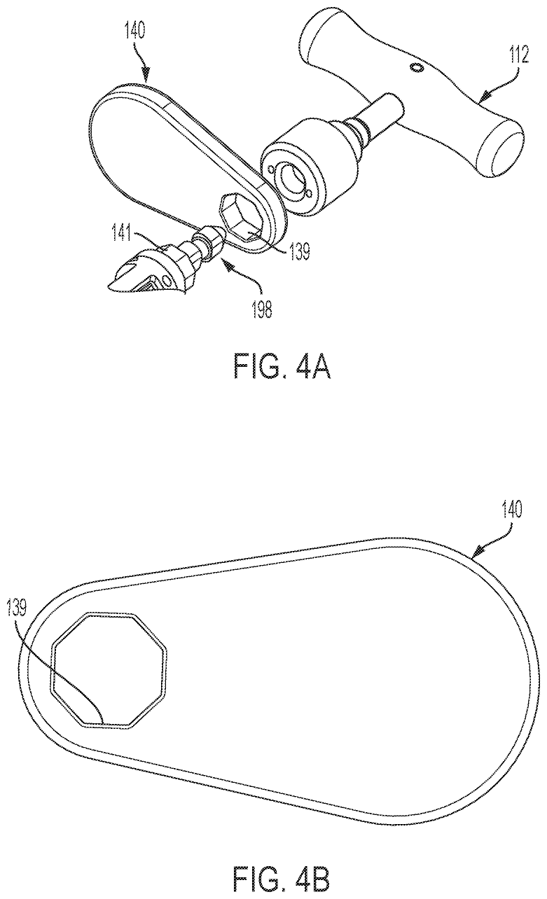

FIGS. 4A and 4B illustrate that the surgical extractor can optionally include a planar laterally projecting strike plate 140 located adjacent the proximal end of the first arm. The strike plate includes a proximal face 140a and a distal face 140b and can include an opening 139 e.g., a polygonal opening, for receiving a correspondingly shaped base 141 e.g., polygonal base, of a quick connect 198. So constructed, the strike plate may be rotatably positioned at different angles, and more specifically at different rotated angles about a longitudinal axis of the surgical extractor or the surgical extractor itself and set at an angle that is most appropriate and comfortable to the user for extraction. By way of example, the polygonal opening in the strike plate and the polygonal base 141 of the quick connect can be octagonal, whereby the strike plate may be placed in any one of eight positions about the surgical extractor, or alternatively a polygonal base with a square, pentagon, hexagon, heptagon, nonagon, decagon, dodecagon shape or any number of sides corresponding in number to a plurality of sides of the polygonal opening. The extraction device 112 holds the strike plate 140 in place and both are easily removable from the quick connect 198.

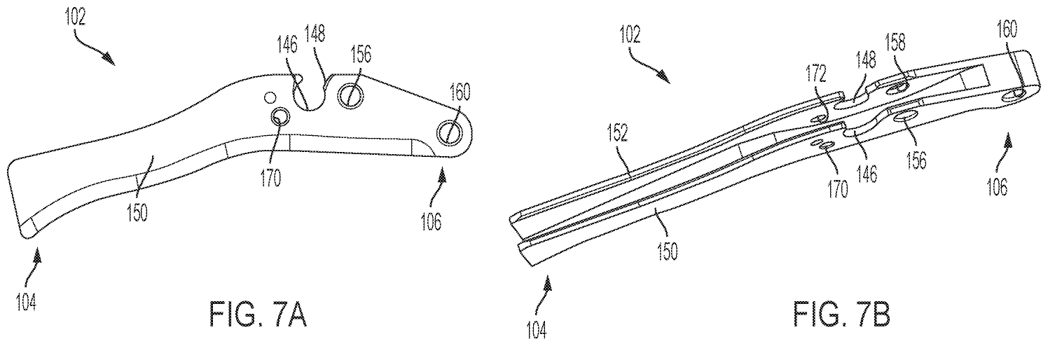

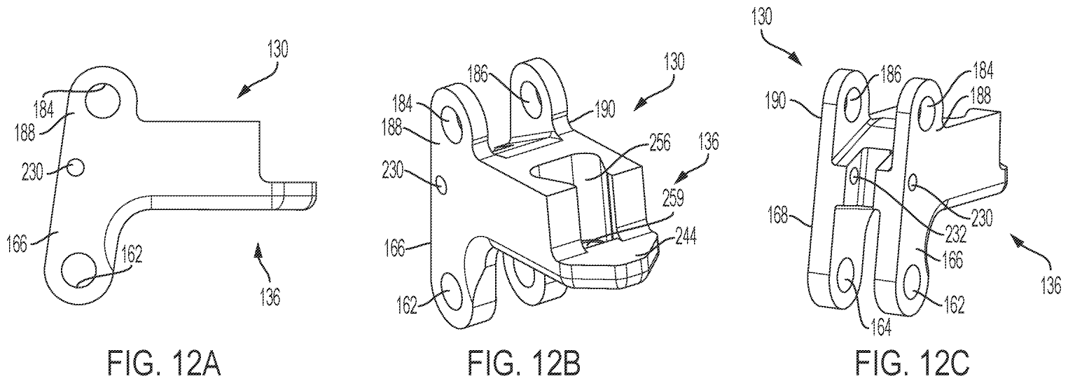

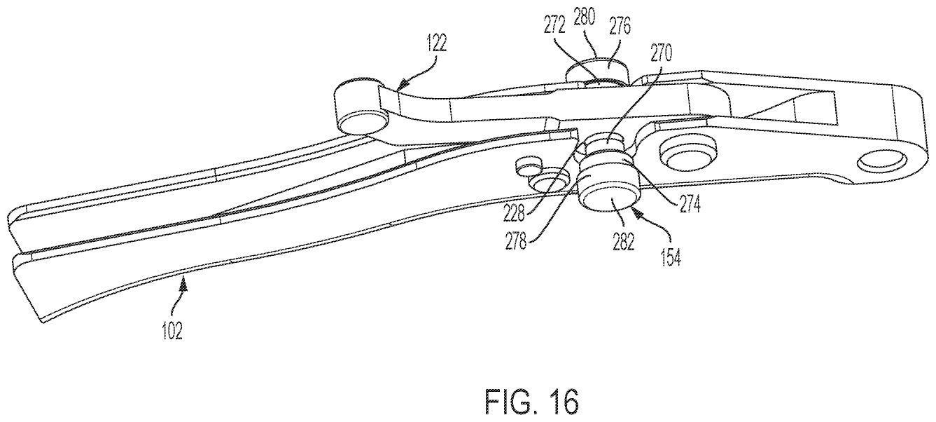

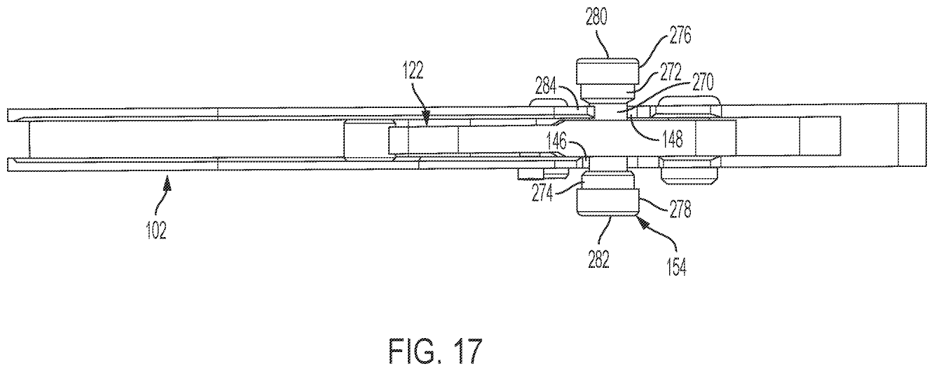

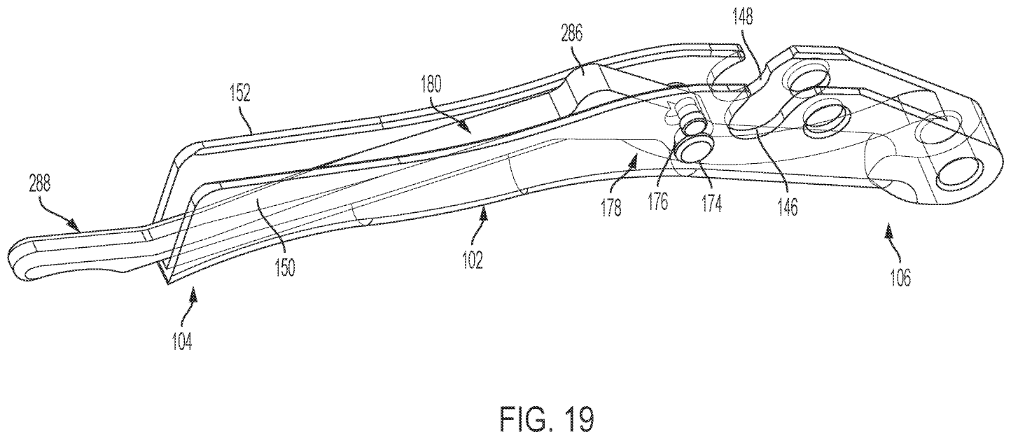

The second arm 102 is configured as best shown in FIGS. 1, 7A and 7B, and is constructed as an elongated arm having a channel formed by upstanding sidewalls 150, 152. About its midportion the second arm is provided with a pair of notches 146, 148 in the upstanding side walls 150, 152 which are adapted to cooperate with a locking mechanism 154 (described below in connection with FIGS. 16 and 17) carried by the lever 122. The side walls 150, 152 further include a first pair of aligned openings 156, 158 that receive the pivot pin 128 for pivotably connecting the distal end 126 of the lever 122 to the second arm. About the distal end 106 of the second arm is a through bore 160 that aligns with aligned openings 162, 164 provided in the lower branches 166, 168 of the link 130 (FIGS. 12A-12C) to receive the pivot pin 134 to pivotably connect the distal end of the second arm 102 to the lower branches of the link. Additionally, the side walls 150, 152 include a second pair of aligned openings 170, 172 that receive a pivot pin 174 (FIGS. 1, 3, and 5) that likewise passes through a through bore 176 provided at a distal end 178 of a release lever 180 (FIG. 19) for pivotably connecting the distal end of the release lever to the second arm. The function of the release lever 180 is described in greater detail in connection with FIG. 19.

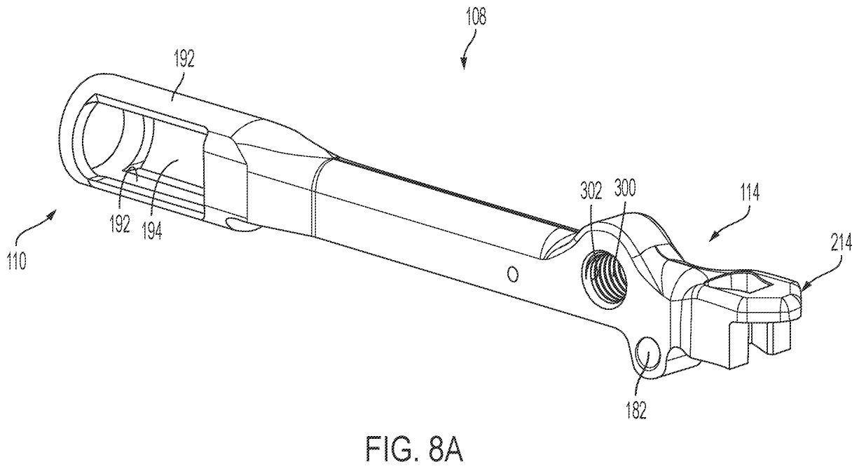

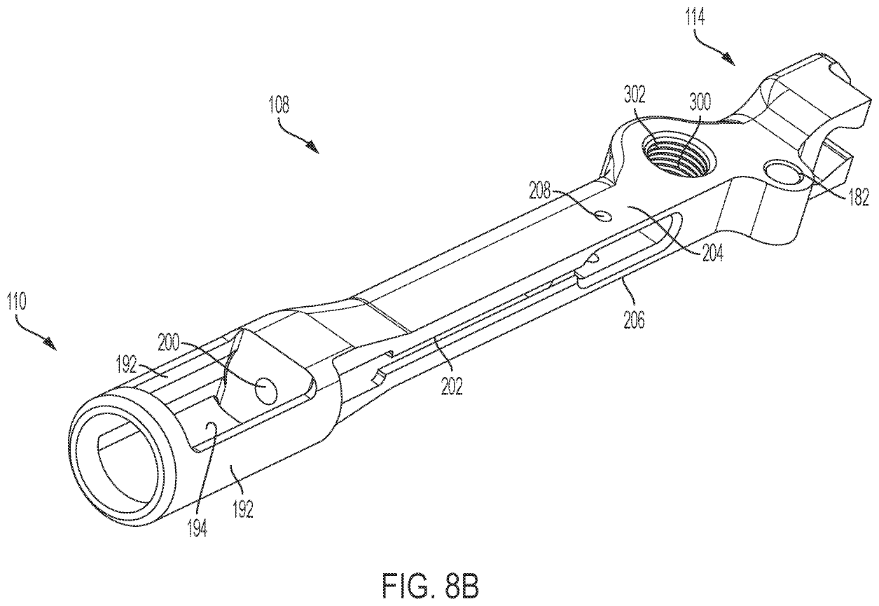

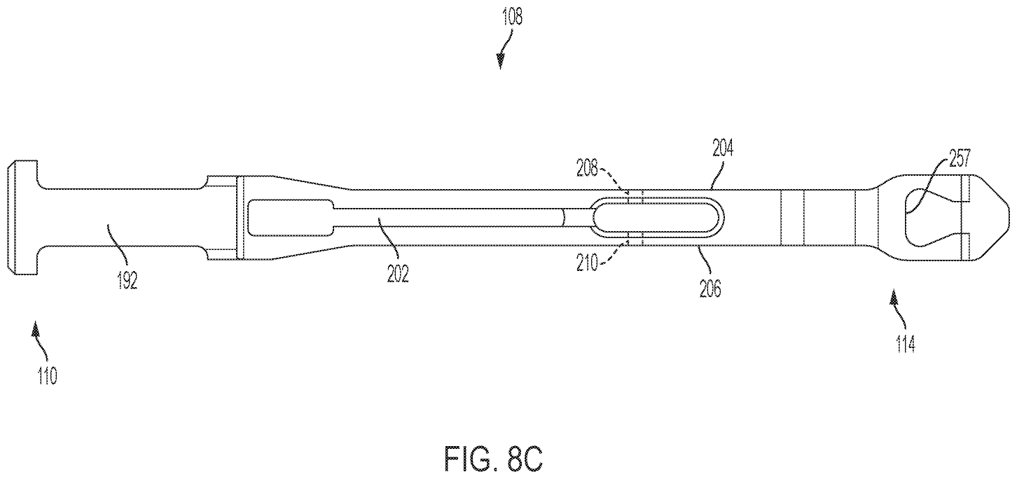

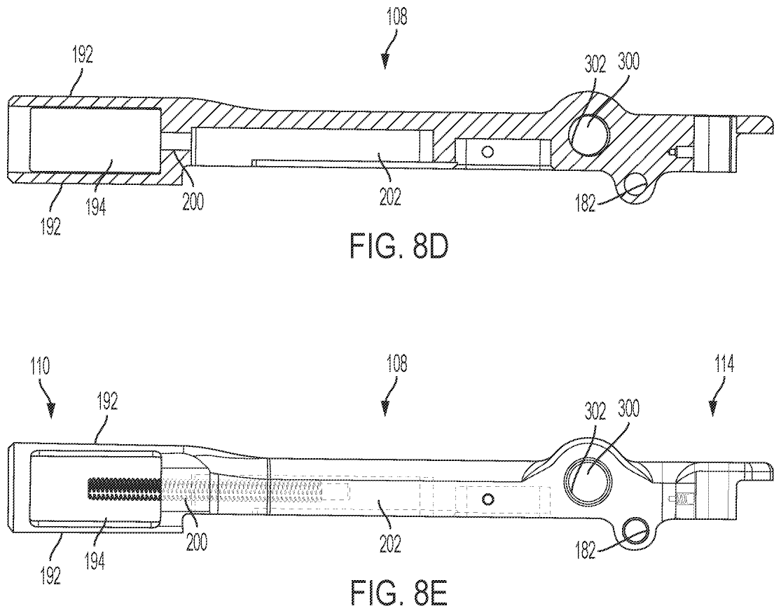

The construction of the first arm 108 is configured as best shown in FIGS. 1, 3, 5 and 8A-8E. FIGS. 8A-8E show a first arm body 109 of the first arm. Adjacent the distal end 114 of the first arm body there is provided a through bore 182 that aligns with aligned openings 184, 186 provided in the upper branches 188, 190 of the link 130

(FIGS. 12A-12C) to receive the pivot pin 132 to pivotably connect the distal end of the first arm 108 to the upper branches of the link. The first arm body includes a fastener 302 configured to releasably retain various forms of extraction devices which are described in greater detail hereinafter. The fastener includes an opening 300 transverse to a longitudinal axis of the first arm. The fastener is spaced from the distal end of the first arm. In particular, the fastener is positioned about a midportion of the first arm, e.g., between the distal end 114 of the first arm for attaching to the first jaw 116 and a proximal end 110 of the first arm.

Near the proximal end 110, the first arm body includes a cage 192 having an opening or hollow interior 194 for housing a rotatable knob 196 which forms part of the adjuster 120 of the adjustment mechanism 118 (see also FIGS. 1, 3, 5 and 10A). Adjacent a distal end of the cage 192 is an internally threaded through bore 200 (FIGS. 8B, 8D and 8E). The through bore 200 is in communication with the cage 192 and with a slot 202 (FIGS. 8C-8F) structured to house a rod 216, as further discussed below. Adjacent the slot 202 the first arm body includes a pair of side walls 204, 206 that is provided with aligned openings 208, 210 (FIGS. 8B and 8C) to receive a pin 212 which holds a first end of a biasing member 144 (FIGS. 1, 3, and 5). As shown in FIG. 8A, at the tip of the distal end 114, the first arm includes a slidable lock 214 for attachment to the first jaw 116, which is discussed in further detail below.

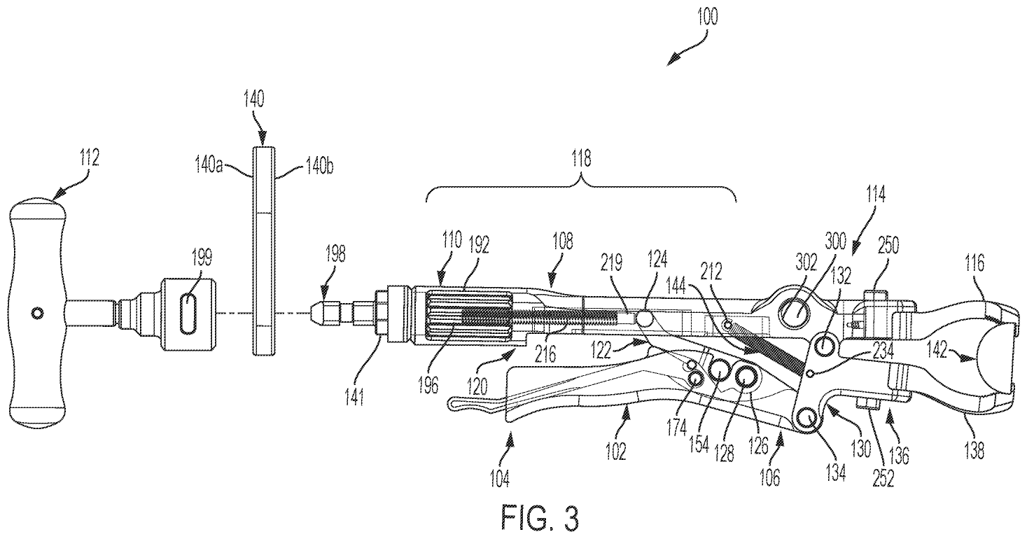

The first arm also includes the quick connect 198 (FIG. 3) about its proximal end structured to releasably engage with, e.g., a corresponding female quick connection carried by the extraction device 112. The corresponding female quick connection includes a biased locking member 199.

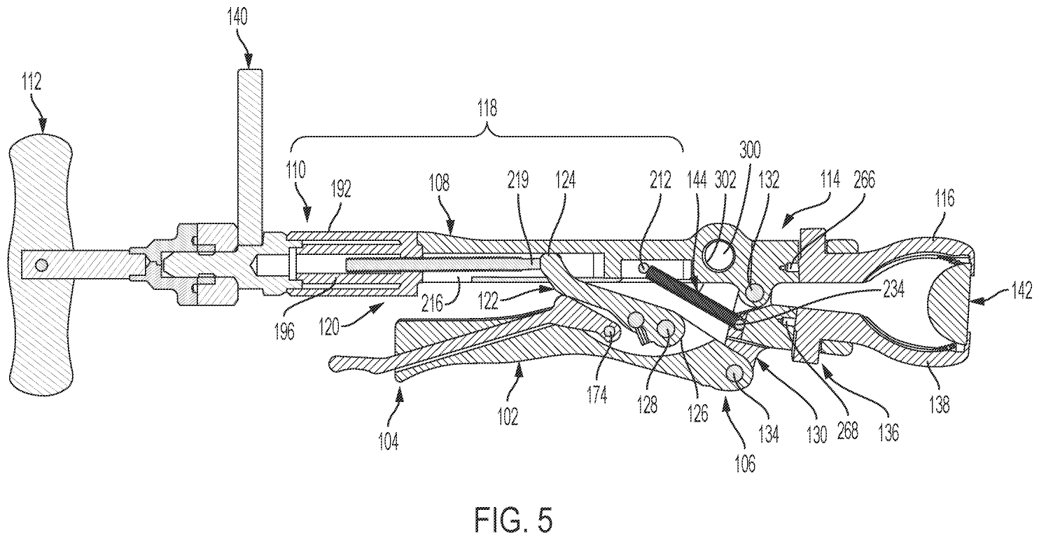

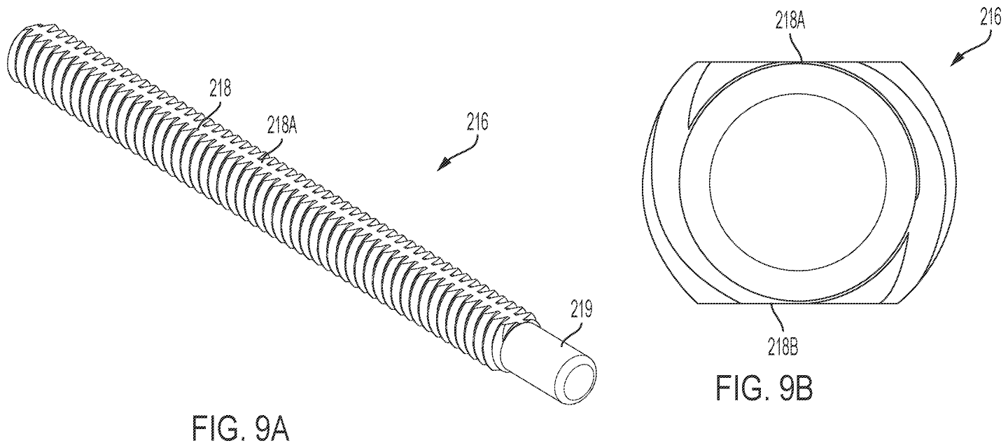

FIGS. 3 and 5 best illustrate the adjustment mechanism 118 of the first arm 108. The adjustment mechanism includes the adjuster 120 and the lever 122. The adjuster comprises the rotatable knob 196 and the rod 216. The rod 216 extends from the rotatable knob and is movable relative to the rotatable knob. The rod 216 is a threaded rod (FIGS. 8E, 9A and 9B) threadedly engaged with the first arm at the internally threaded through bore 200 as shown in FIG. 8E. The rod 216 includes at least one planar side 218, and preferably a pair of opposing planar sides 218A, 218B. The rod also includes a non-threaded distal nose 219. The rod is sized in length sufficiently such that the distal nose 219 of the rod 216 abuts the proximal end 124 of the lever 122.

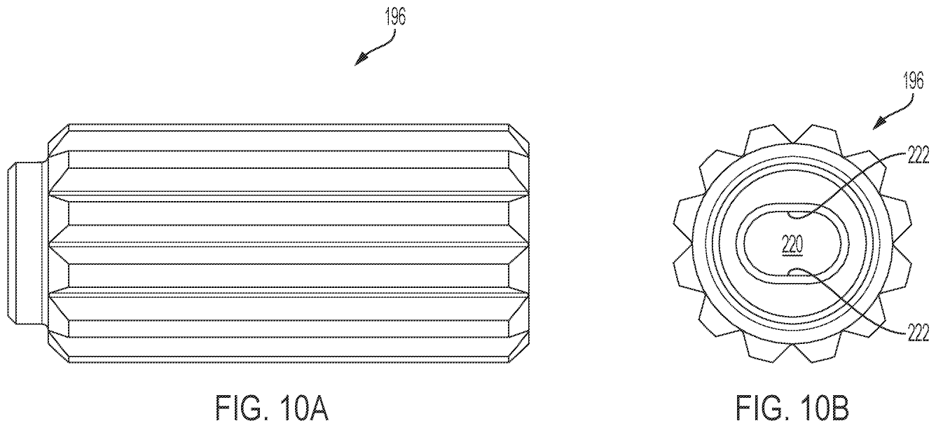

The rotatable knob 196 is configured as best shown in FIGS. 10A and 10B having a generally cylindrical configuration. The outer surface of the rotatable knob is preferably textured, e.g. with splines, knurling or the like, to enhance gripping of a user's fingers when rotating the rotatable knob. As shown in FIG. 10B, the rotatable knob 196 has an opening 220 about its end or proximally facing end, with a planar side 222 to cooperate with the planar side 218 of the rod 216. Rotation of the rotatable knob 196 causes rotation of the threaded rod 216 within the internally threaded through bore 200, thereby causing the rod 216 to extend from or retract into the rotatable knob depending on the direction of rotation of the rotatable knob, or in other words move along a direction of a longitudinal axis of the rod. More particularly, rotation of the rotatable knob 196 in a first direction causes the rod 216 to extend from the rotatable knob. In so doing, the distal nose 219 of the rod pushes further against the proximal end 124 of the lever 122. Conversely, rotation of the rotatable knob 196 in a second direction causes the rod 216 to retract into the rotatable knob. In so doing, the proximal end 124 of the lever 122 remains in contact with the distal nose 219 of the rod 216 since it is under the influence of the biasing member 144.

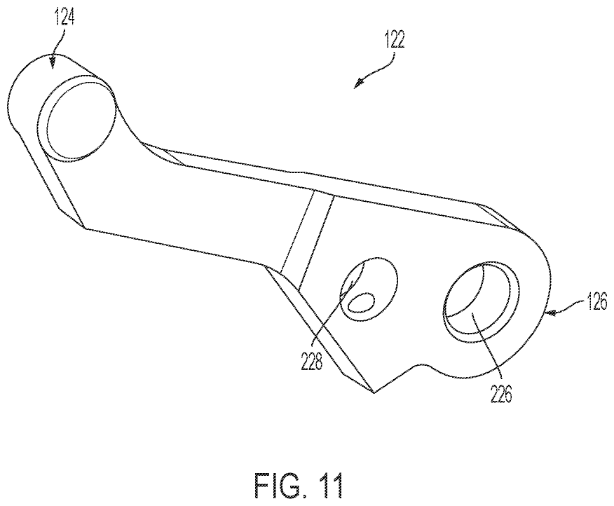

The lever 122 is structured as best shown in FIGS. 1, 5 and 11. Referring to FIG. 11, between its proximal and distal ends 124, 126, the lever 122 includes a first through bore 226 that receives the pivot pin 128 for pivotably connecting the distal end 126 of the lever 122 to the second arm (FIGS. 3, 5 and 6). The lever 122 further includes a second through bore 228 through which the locking mechanism 154 passes (see FIGS. 1-3, 6 and 16).

FIGS. 12A-12C best show the construction of the link 130. The link includes upper and lower branches. Between the upper branches 188, 190 and lower branches 166, 186, the link includes a pair of aligned through bores 230, 232. The through bores 230, 232 receive a pin 234 which holds a second end of the biasing member 144 (FIGS. 1, 3, and 5). The distal end 136 of the link 130 is provided with a fastener 256 for attaching the second jaw 138 to the link. Likewise, the distal end 114 of the first arm 108 is provided with a fastener 254 for attaching the first jaw 116 to the first arm. As shown in FIG. 12B the link includes an aperture 259 for receiving a mail dovetail 252 of the second jaw 138. As a result the first jaw is releasably attachable to the distal end of the first arm via engagement of a male dovetail 250 of the first jaw with an aperture 257 of the first arm and the second jaw is releasably attachable to the distal end of the link via engagement of the male dovetail 252 with the link aperture 259.

The fasteners at the distal ends of the link and the first arm are preferably cooperating fasteners or slidable locks that respectively engage cooperating fasteners or cooperating slidable locks provided at the proximal ends of the first and second jaws.

The surgical extractor 100 further comprises the aforementioned biasing member 144 which is connected to and biases the link 130 and the first arm 108, in a manner described in greater detail below. The biasing member can be e.g., a tension spring, an elastomer or the like. In the present embodiment, the tension spring has a spring constant of about 0.5 to 8.0 lbs, including 0.4, 0.6, 0.7, 0.8, 0.9, 1.0, 2.0, 3.0, 4.0, 5.0, 6.0, 7.0, and 9.0 lbs.

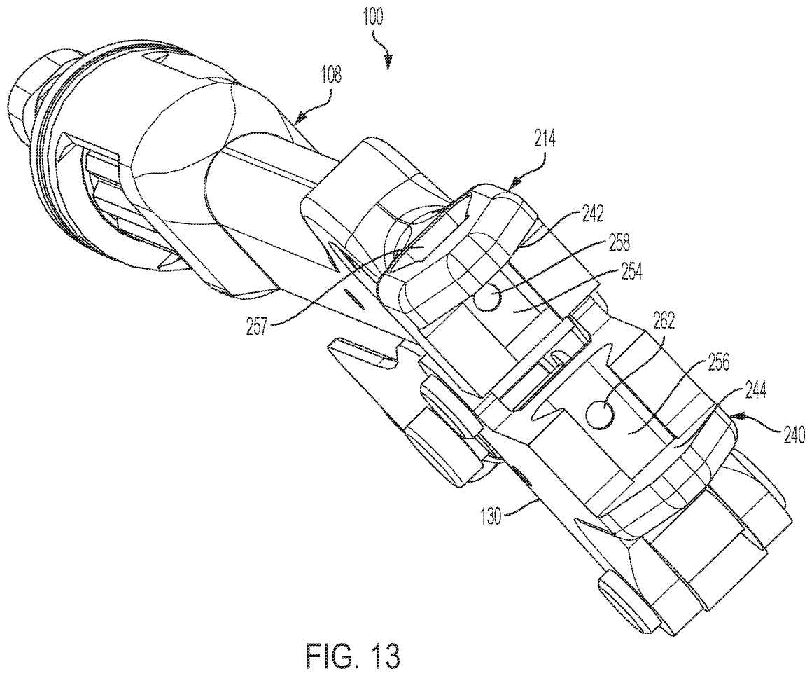

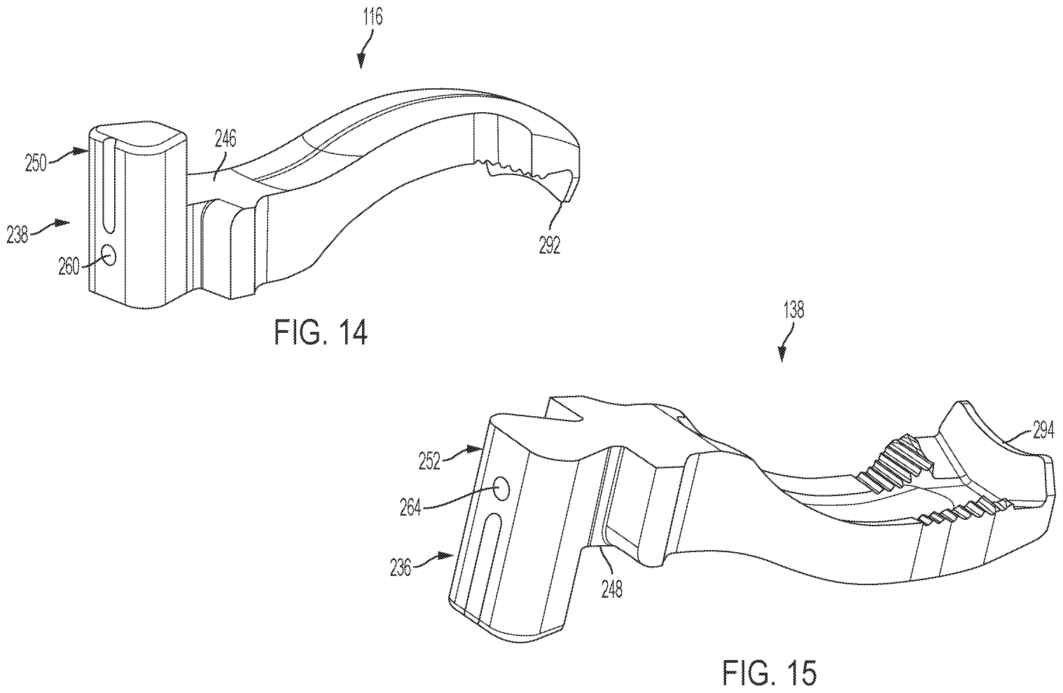

The first and second jaws respectively are structured as best shown in FIGS. 14 and 15. The first and second jaws respectively include a corresponding recess 260 and 264 to releasably engage respective corresponding detents 258, 262 (FIG. 13) on the distal ends of the first arm and the link. The first and second jaws respectively include slidable locks 236 and 238 to slidingly engage a corresponding slidable lock 214, 240 on the first arm 108 and link 130, respectively, as shown in FIG. 13. Referring to FIG. 13, the corresponding slidable locks on the first arm and link each includes a stop 242, 244. According to an aspect, the stops 242, 244 are distally extending ledges. The stops 242, 244 are engageable by flats 246, 248 respectively provided on the first and second jaws (FIGS. 14 and 15) to limit insertion of the first and second jaws into the first arm and the link.

According to another aspect, the slidable lock 236 and 238 on each of the first and second jaws is the male dovetail 250 and 252, respectively (FIGS. 14 and 15), and the corresponding slidable lock 214, 240 on each of the first arm and the link is a female dovetail 254 and 256, respectively (FIG. 13). The first and second jaws are thus inserted from inner or medial sides of the first arm and the link towards their outer or lateral sides thereof, whereby the flats 246, 248 stop the slidable locks from further insertion upon engagement with the stops 242, 244. Additionally, the male dovetails 250, 252 of the jaws have longitudinal lengths whereby, when inserted into the female dovetails 254, 256, the male dovetails project outwardly of the first arm and the link, as shown in FIGS. 1 and 3.

According to another aspect, the implant extractor further comprises a detent 258 (FIG. 13) carried by one of the first jaw and the first arm, or a detent 262 carried by the one of the second jaw and the link. In the illustrated exemplary embodiment, the detent 258 is carried by the first arm for engaging a corresponding recess 260 in the first jaw, and the detent 262 is carried by the link for engaging corresponding recess 264 in the second jaw (FIGS. 13-15). An exemplary detent can be e.g., a ball detent. As shown in FIG. 5, the detents 258, 262 are urged outwardly toward engagement with recesses 260, 264 by biasing members 266 and 268, such as springs or the like. Engagement of the detents with their corresponding recesses operates to resist inadvertent dislodgement of the first and second jaws from the first arm and the link. When it is desired to release the jaws from the first arm and the link, the user presses against the outwardly projecting male dovetails 250, 252 with sufficient force to overcome the biasing force of the biasing members 266, 268, whereby the detents become dislodged from the recesses.

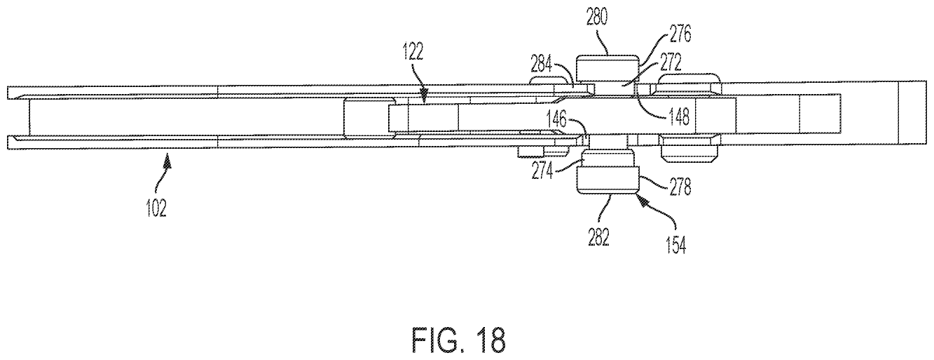

The locking mechanism 154 is configured as best shown in FIGS. 16-18. The locking mechanism 154 is carried by the lever 122. That is, the locking mechanism is on the lever and movable between a locked position and an unlocked position. In the locked position (FIG. 18), the locking mechanism maintains clamping engagement of the first and second jaws and, in the unlocked position (FIGS. 16 and 17), the locking mechanism permits release of the first and second jaws from clamping engagement with an object to be extracted.

The locking mechanism includes a central shaft 270 sized to reciprocate within the through bore 228 of the lever 122. At opposite ends of the central shaft 270, the locking mechanism includes first cylindrical portions 272, 274 of larger diameter than the central shaft that are sized to be received in notches 146, 148 provided in the upstanding side walls of the second arm 102. Additionally, adjacent the outside or lateral ends of the first cylindrical portions are second cylindrical portions or buttons 276, 278 of larger diameter than the first cylindrical portions. The outer surfaces 280, 282 of the buttons 276, 278 are adapted to be pressed by a user's finger.

To place the locking mechanism 154 into the locked position, the user presses the outer surface 280 of the button 276 until the first cylindrical portion 272 is received in notch 148 (FIG. 18). The notch 148 includes an overhang 284 which overlies the first cylindrical portion 272. The overhang prevents dislodgement of the cylindrical portion 272 from the notch 148 and secures the second arm 102 and the lever 122 connected thereto into a locked position.

To place the locking mechanism 154 into the unlocked position, i.e., to release the locking mechanism from the locked position, a user presses the outer surface 282 of the button 278 until the first cylindrical portion 272 is no longer received in the notch 148 and retained by the overhang 284. With the locking mechanism 154 in such position, the user can separate the second arm from the first arm whereby the first and second jaws are released from clamping engagement with, e.g., implant 142.

The implant extractor 100 further comprises a release lever 180 (FIG. 19) on the second arm 102 to release the first and second jaws from clamping engagement with an implant to be extracted. The release lever has a knuckle 286 provided adjacent the distal end 178 thereof, and a proximal end 288 projecting from the proximal end 104 of the second arm. Before deploying the release lever, the user must place the locking mechanism 154 oriented in the unlocked position. The proximal end 288 of the release lever is lifted upwardly (as shown in FIG. 19) until the knuckle 286 contacts the underside of the lever 122. Further lifting of the release lever operates to withdraw the locking mechanism from the notches 146, 148 and push the second arm 102 away from the first arm 108, thereby allowing the first and second jaws to spread apart.

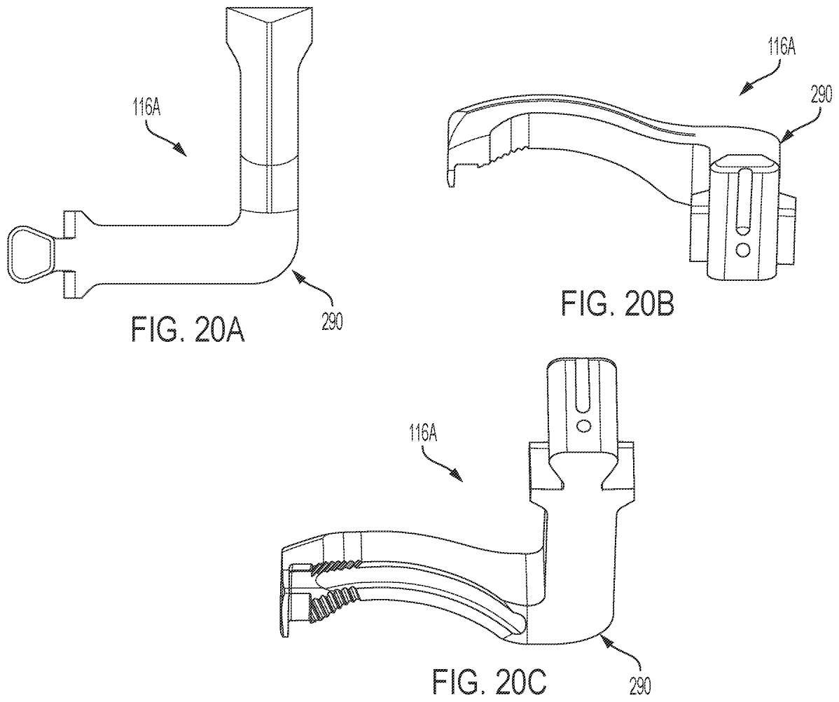

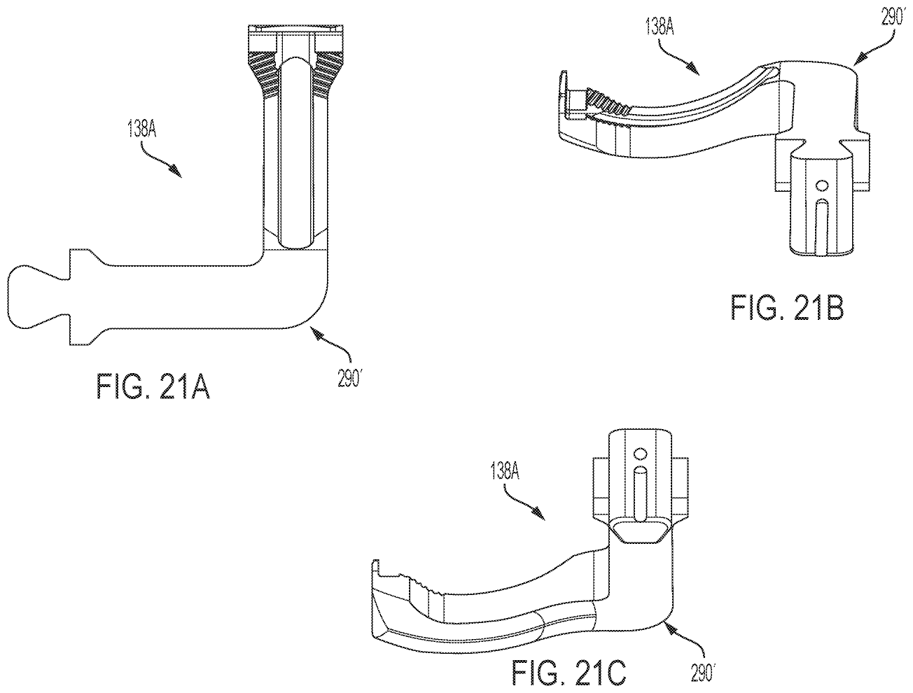

The first and second jaws 116, 138 heretofore discussed can be referred to as straight jaws in that the proximal ends of the jaws extend straight from the distal ends of the first arm and the link. FIGS. 20A-20C and 21A-21C illustrate an alternative exemplary configuration of the first and second jaws. The first and second jaws 116A, 138A illustrated can be referred to as laterally offset jaws. In this regard, intermediate regions of the first and second jaws 116A, 138A include laterally directed bend 290 and 290′, respectively, whereby the distal ends of the jaws are laterally offset from the distal ends of the first arm and the link.

The first and second jaws 116, 138 (or 116A, 138A) are operable to releasably clamp a medical implant including, without limitation, e.g., a glenosphere implant 142. As will be described hereinafter, the first and second jaws can assume other configurations suitable for extracting objects such as implants and surgical hardware from the body including, without limitation, intramedullary nails, rods and wires, bone screws, surgical hex nuts, revision sleeves, femoral head implants, glenosphere implants and other implants.

Referring to the first and second jaws 116, 138 as an example, in order to clamp the first and second jaws onto an implant to be extracted, a user first rotates the rotatable knob 196 in a first direction which causes the first and second jaws to separate until opposed lips 292, 294 at respective distal ends of the first and second jaws (FIGS. 14 and 15) are spaced slightly wider than the circumference of the implant to be extracted. More particularly, rotation of the rotatable knob in the first direction causes the distal end 126 of the lever and the second arm 102 to move rearwardly, whereby the link 130 pivots rearwardly and the second jaw 138 moves away from the first jaw 116. The user then places the opposed lips adjacent the implant and rotates the rotatable knob in the opposite direction which causes the first and second jaws to close around the implant until the lips 292, 294 are positioned behind the implant. More particularly, rotation of the rotatable knob in the opposite direction causes the distal end 126 of the lever and the second arm 102 to move forwardly, whereby the link 130 pivots forwardly causing the second jaw 138 to move toward the first jaw 116. Additionally, the biasing member 144 keeps the first and second jaws open during clamping of the implant 142. That is, the biasing member serves to prevent the second jaw from uncontrolled movement which could hinder clamping of the first and second jaws to the implant. The user then squeezes the first and second arms together whereupon the second arm pivots posteriorly until the locking mechanism 154 becomes seated in the notches 146, 148 of the second arm. During seating of the locking mechanism into the notches, the first and second jaws are urged into tight clamping engagement with the implant 142. Once the locking mechanism is fully seated in the notches, the user presses the outer surface 280 of the button 276 until the first cylindrical portion 272 is received in the notch 148, thereby locking the position of the second arm relative to the first arm. With the second arm locked and the surgical extractor 100 secured to the implant, the user may use the surgical extractor to pull the implant from the bone to which it is attached. If additional force is necessary to extract the implant, the user may strike the distal face 140b of the strike plate 140 with a hammer, mallet or similar striking tool to dislodge the implant. Once the implant is freed, the user unlocks the locking mechanism 154 and lifts the proximal end 284 of the release lever to open the first and second jaws and release the implant from the surgical extractor.

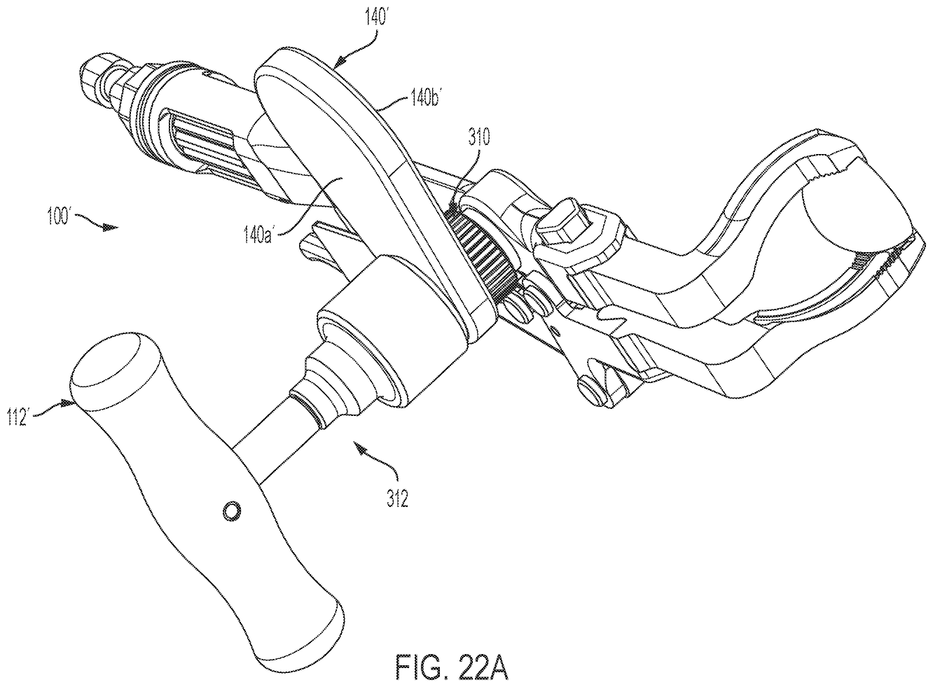

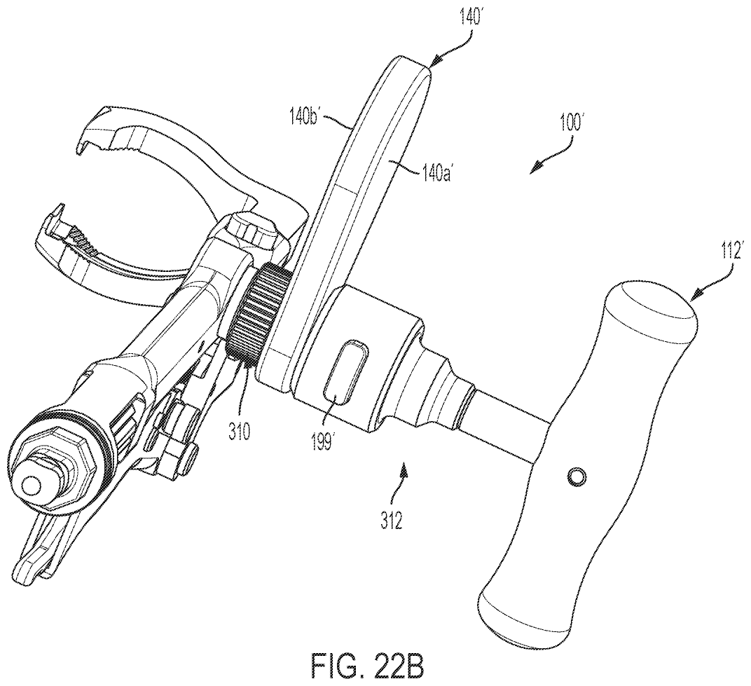

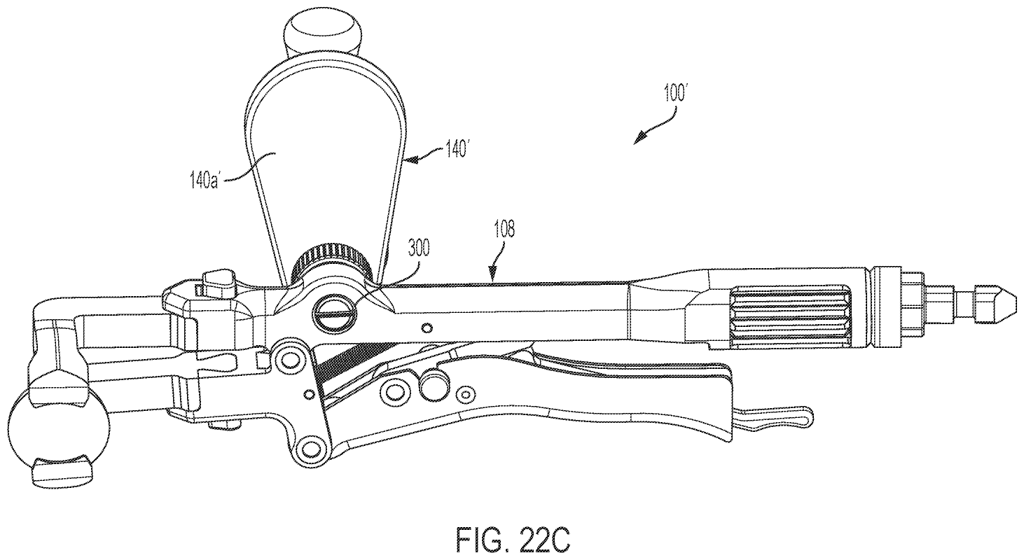

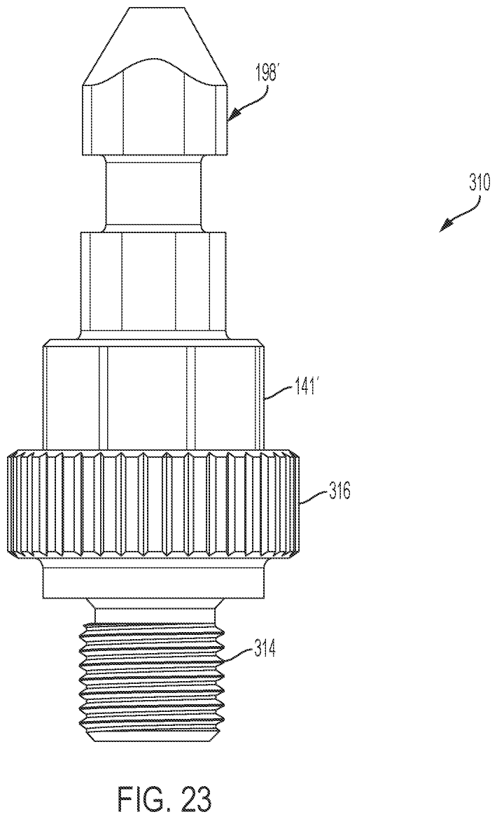

FIGS. 22A-22C show another exemplary embodiment of a surgical extractor 100′ according to the subject disclosure. In many respects, the surgical extractor 100′ is similar in construction to the surgical extractor 100. Accordingly, for brevity only those aspects of the surgical extractor 100′ which materially depart in structure and/or function from the surgical extractor 100 will be described in detail. In this regard, the surgical extractor 100′ comprises an extraction device connector 310 (FIG. 23) constructed and arranged to releasably join an extraction device 312 to the fastener 302 of the first arm 108. In the illustrated example of FIGS. 22A-22C, the extraction device connector extends laterally away from the first arm when connected to the fastener on the opposite side as the distal ends of the laterally extending jaws. According to an aspect, the extraction device 312 comprises a T-handle 112′ and a strike plate 140′ (including an upper face 140a′ and a lower face 140b′) which are releasably connectable to the connector member 310 similar to the manner in which the T-handle and the strike plate are joined to the proximal end of the surgical extractor 100, as described above.

Referring to FIG. 23, the extraction device connector 310 includes a cooperating fastener 314 in the form of external threading at a first end thereof for cooperatively engaging the corresponding fastener in the form of internal threading 302 in the transverse opening 300 of the first arm 108. While illustrated as being threading, the cooperating fastener of the connector member and the fastener of the first arm may assume other forms including, without limitation, a J-slot connection, a press-fit, a Luer lock, a snap fit, a ball detent, and the like. Adjacent threading 314, the connector member further includes a radially projecting turning knob 316, the exterior of which is desirably provided with grip-enhancing structure such as knurling or, as illustrated, ribs to facilitate threading of the connector member into the transverse opening in the first arm. On the side of knob 316 opposite fastener structure 314 is a base 141′ for receiving an unillustrated correspondingly shaped opening in the strike plate 140′. The base can be e.g., polygonal in shape, or assume any other shape suitable for receiving the opening in the strike plate. The connector member further includes a quick connect 198′ that is configured to releasably engage with a corresponding quick connect, e.g., a biased locking member 199′ (FIG. 22B) carried by the extraction device 312. The T-handle 112′ holds the strike plate 140′ in place and both are easily removable from the quick connect 198′ upon depression of the biased locking member 199′.

When the extraction device 312 is secured to the surgical extractor 100′ and the surgical extractor is clamped on an implant in the manner described above, a user may strike the lower face 140b′ of the strike plate 140′ with a hammer, mallet or the like, in order to dislodge the implant from bone.

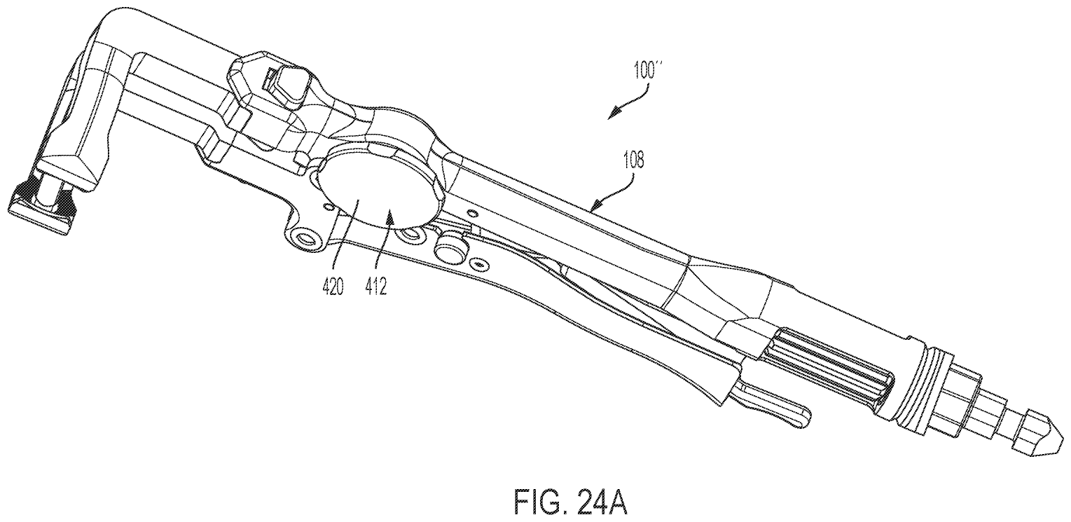

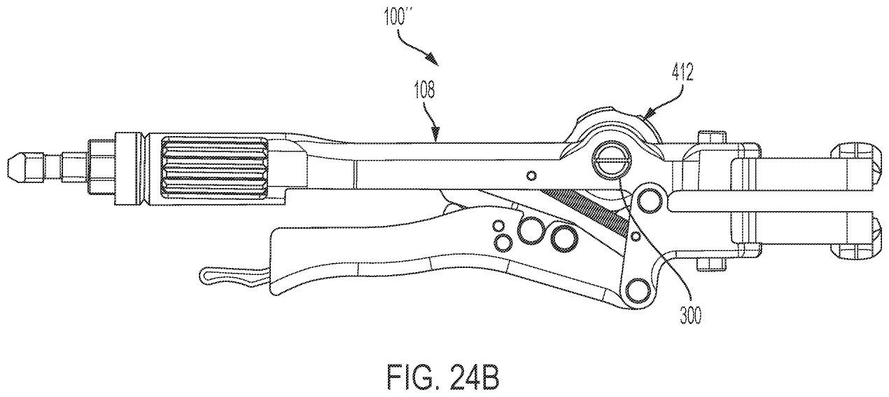

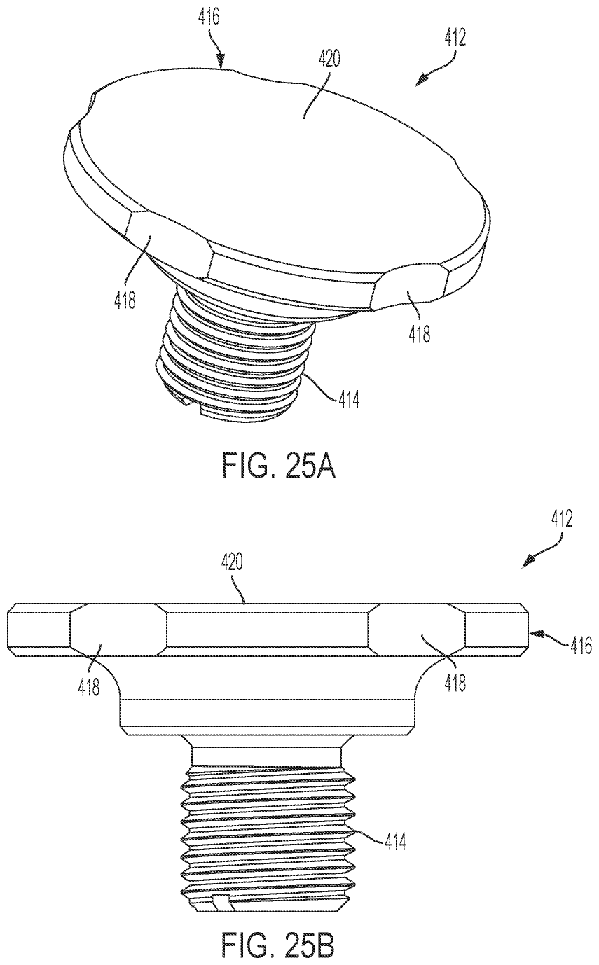

Referring to FIGS. 24A and 24B, there is shown another exemplary embodiment of a surgical extractor 100″ according to the subject disclosure. Since the surgical extractor 100″ is structurally and functionally similar to surgical extractor 100, only those aspects of the surgical extractor 100″ which materially depart in structure and/or function from the surgical extractor 100 will be described in detail. In this regard, the surgical extractor 100″ includes an extraction impactor 412. In the illustrated example, the extraction impactor extends laterally away from the first arm when connected to the fastener on the same side as the distal ends of the laterally extending jaws. As shown in FIGS. 25A and 25B, the extraction impactor includes a cooperating fastener 414 in the form of external threading for engaging the corresponding fastener in the form of internal threading 302 of the transverse opening 300 in the first arm 108. While illustrated as being cooperating threading, the cooperating fastener of the extraction impactor and the fastener in the first arm may assume other forms including, without limitation, a J-slot connection, a press-fit, a Luer lock, a snap fit, a ball detent, and the like. Adjacent threading 414, the extraction impactor further includes a radially projecting turning knob 416, the periphery of which is desirably provided with grip-enhancing structure such as a plurality of notches 418 for receiving a user's fingers to facilitate threading of the extraction impactor 412 into the transverse opening in the first arm. The exposed upper surface 420 defines a striking surface adapted for striking by a hammer, mallet or the like.

When the extraction impactor 412 is secured to the surgical extractor 100″ and the surgical extractor is clamped on an implant in the manner described above, a user may strike the upper surface 420 of the extraction impactor with a hammer, mallet or the like, in order to dislodge the implant from bone.

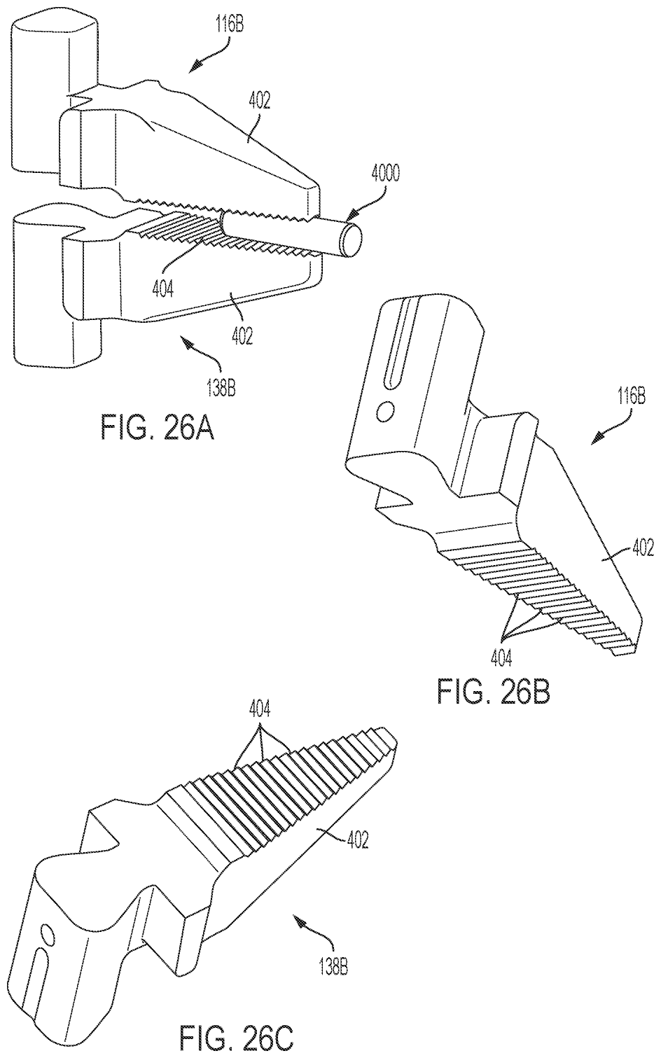

FIGS. 26A through 37B illustrate exemplary, although non-limitative, releasable modular jaws suitable for use with a surgical extractor according to the subject disclosure. Similar to those described above in connection with jaws 116 and 138, the slidable locks on the jaws shown in FIGS. 26A through 37B are configured as male dovetails adapted to be received in female dovetails provided in the first arm 108 and the link 130 of the surgical extractor 100 (FIG. 1). Likewise, the male dovetails of the releasable jaws shown in FIGS. 26A through 37B are provided with recesses for receiving detents carried by the first arm and the link. The slidable locks and the cooperating recesses and detents structure and their modes of operation have been described in detail above. Accordingly, for brevity, such description will not be repeated in connection with the releasable jaws illustrated in FIGS. 26A through 37B.

Referring to FIGS. 26A-26C there is shown a pair of jaws 116B, 138B which are attachable to the first arm 108 and link 130, respectively (FIG. 1). Jaws 116B, 138B are suitable for gripping an end of an elongated object 4000 such as a wire or pin and each include a bite portion 402 which tapers from a proximal end to a distal end thereof. Opposing gripping faces of the bite portions are provided with a plurality of transversely extending teeth or ridges 404 for firmly gripping the object.

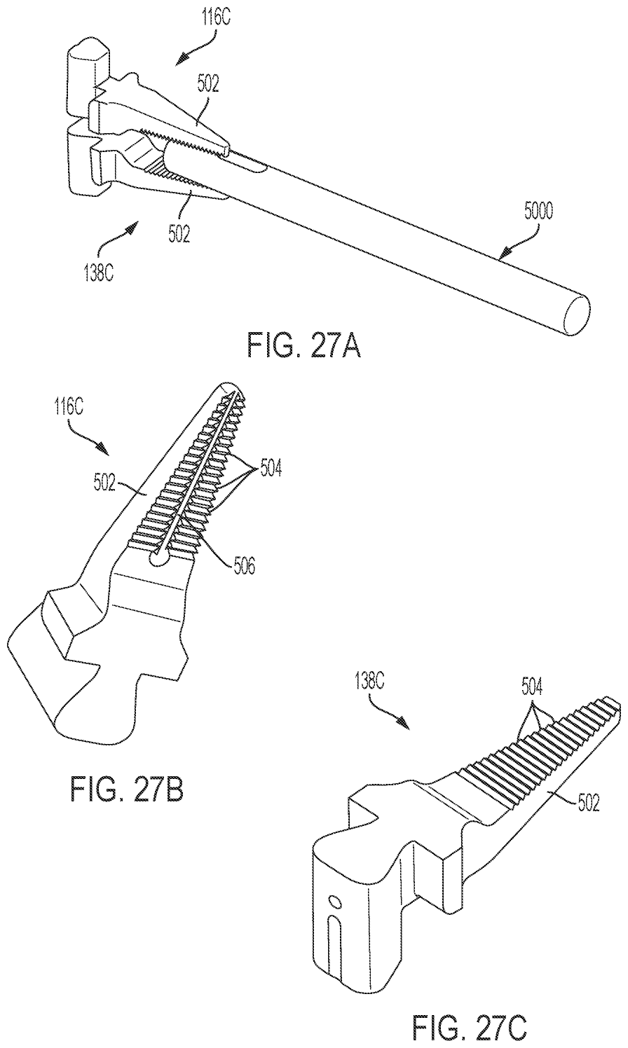

Referring to FIGS. 27A-27C there is shown a pair of jaws 116C, 138C which are attachable to the first arm 108 and link 130, respectively (FIG. 1). Jaws 116C, 138C are suitable for gripping an end of an elongated object 5000 such as an intramedullary nail and each include a bite portion 502 which tapers from a proximal end to a distal end thereof. Opposing gripping faces of the bite portions are provided with a plurality of transversely extending teeth or ridges 504 for firmly gripping the object. To further enhance gripping of the object, at least one of the jaws (in the illustrated example jaw 116C) can include a substantially centrally disposed longitudinal groove 506 separating the plurality of teeth. Provision of such a groove increases the number of teeth edges which engage the object 5000.

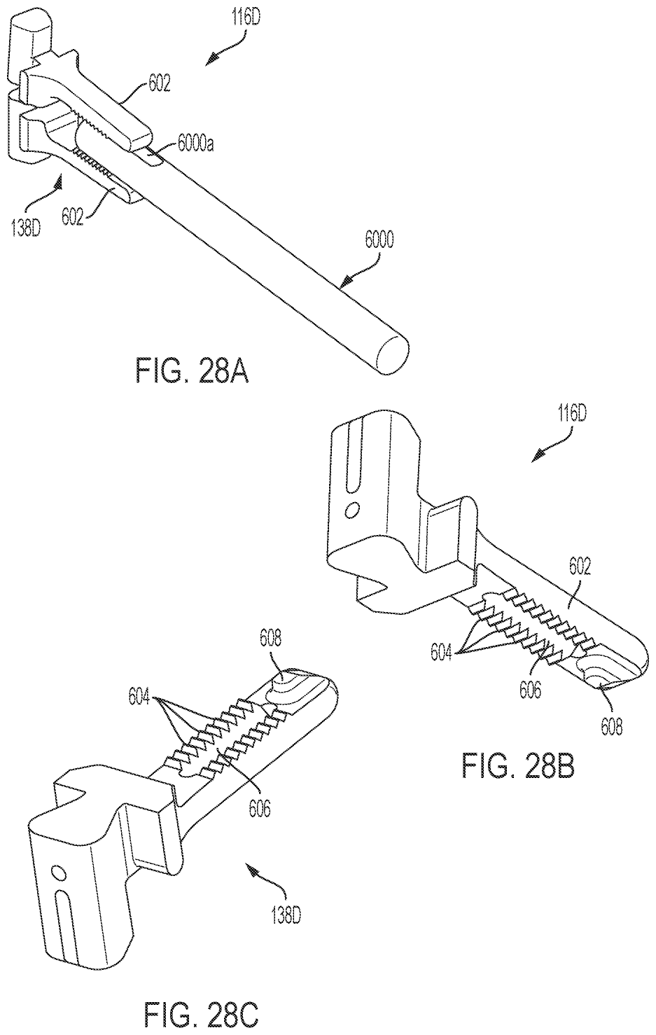

Referring to FIGS. 28A-28C there is shown a pair of jaws 116D, 138D which are attachable to the first arm 108 and link 130, respectively (FIG. 1). Jaws 116D, 138D are suitable for gripping an end of an elongated object 6000 such as a slotted intramedullary nail and each include a bite portion 602. Opposing gripping faces of the bite portions are provided with a plurality of transversely extending teeth or ridges 604 for firmly gripping the object. To further enhance gripping of the object, at least one of the jaws (in the illustrated example both jaws 116D and 138D) can include a substantially centrally disposed longitudinal groove 606 separating the plurality of teeth. Additionally, the distal end of each jaw is provided with an enlarged tooth or projection 608 which stands proud of the gripping face and is configured to engage a slot or similar recess 6000a provided in the object 6000.

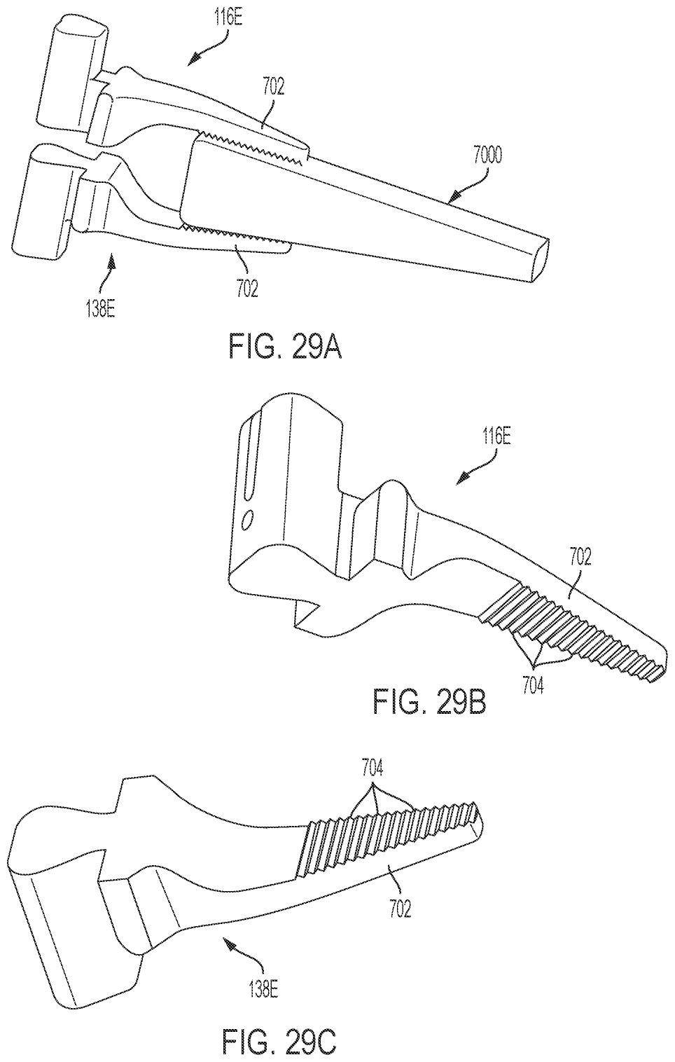

Referring to FIGS. 29A-29C there is shown a pair of jaws 116E, 138E which are attachable to the first arm 108 and link 130, respectively (FIG. 1). Jaws 116E, 138E are suitable for gripping an end of an elongated object 7000 such as a large hip implant and each include a bite portion 702 which tapers from a proximal end to a distal end thereof. Opposing gripping faces of the bite portions are provided with a plurality of transversely extending teeth or ridges 704 for firmly gripping the object.

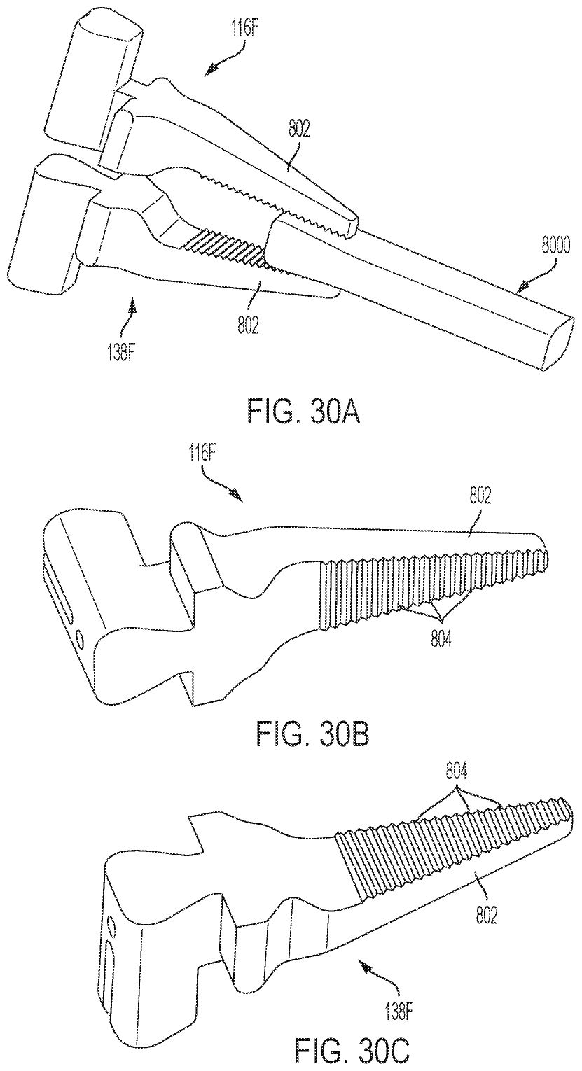

Referring to FIGS. 30A-30C there is shown a pair of jaws 116F, 138F which are attachable to the first arm 108 and link 130, respectively (FIG. 1). Jaws 116F, 138F are suitable for gripping an end of an elongated object 8000 such as a small hip implant and each include a bite portion 802 which tapers from a proximal end to a distal end thereof. Opposing gripping faces of the bite portions are provided with a plurality of transversely extending teeth or ridges 804 for firmly gripping the object.

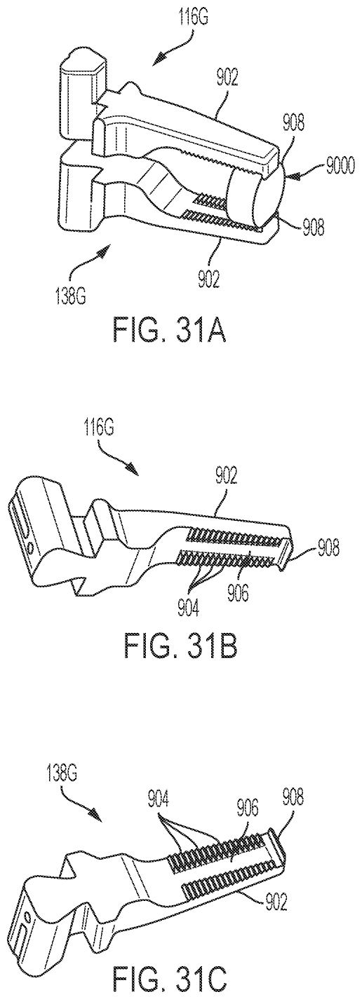

Referring to FIGS. 31A-31C there is shown a pair of jaws 116G, 138G which are attachable to the first arm 108 and link 130, respectively (FIG. 1). Jaws 116G, 138G are suitable for gripping an object 9000 such as a radial head implant and each include a bite portion 902 which tapers from a proximal end to a distal end thereof. Opposing gripping faces of the bite portions are provided with a plurality of transversely extending teeth or ridges 904 for firmly gripping the object. To further enhance gripping of the object, at least one of the jaws (in the illustrated example both jaws 116G and 138G) can include a substantially centrally disposed longitudinal groove 906. Additionally, the distal end of each jaw is provided with a lip-like projection 908 which stands proud of the gripping face and is configured to engage a distal face of the object 9000.

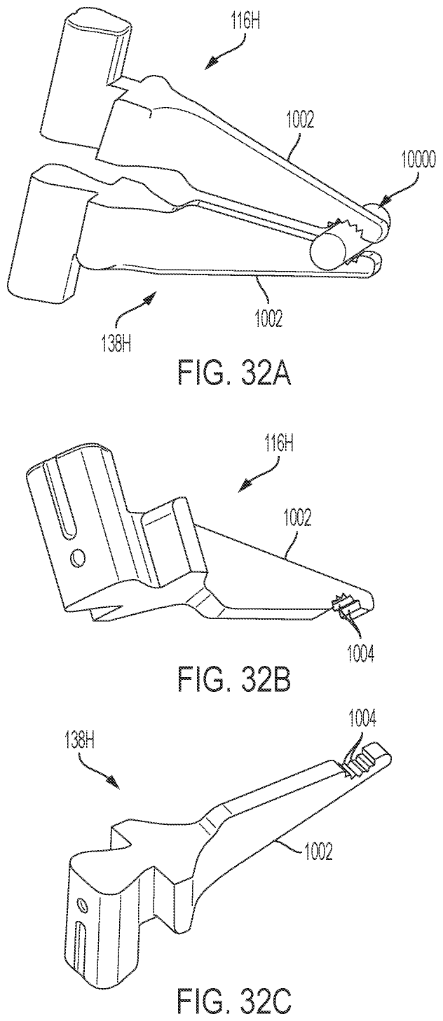

Referring to FIGS. 32A-32C there is shown a pair of jaws 116H, 138H which are attachable to the first arm 108 and link 130, respectively (FIG. 1). Jaws 116H, 138H are suitable for gripping an elongated object 10000 such as a spine rod and each include a bite portion 1002. Opposing gripping faces of the bite portions are provided with a plurality of transversely extending teeth or ridges 1004 arranged in a concave arc for firmly gripping the object.

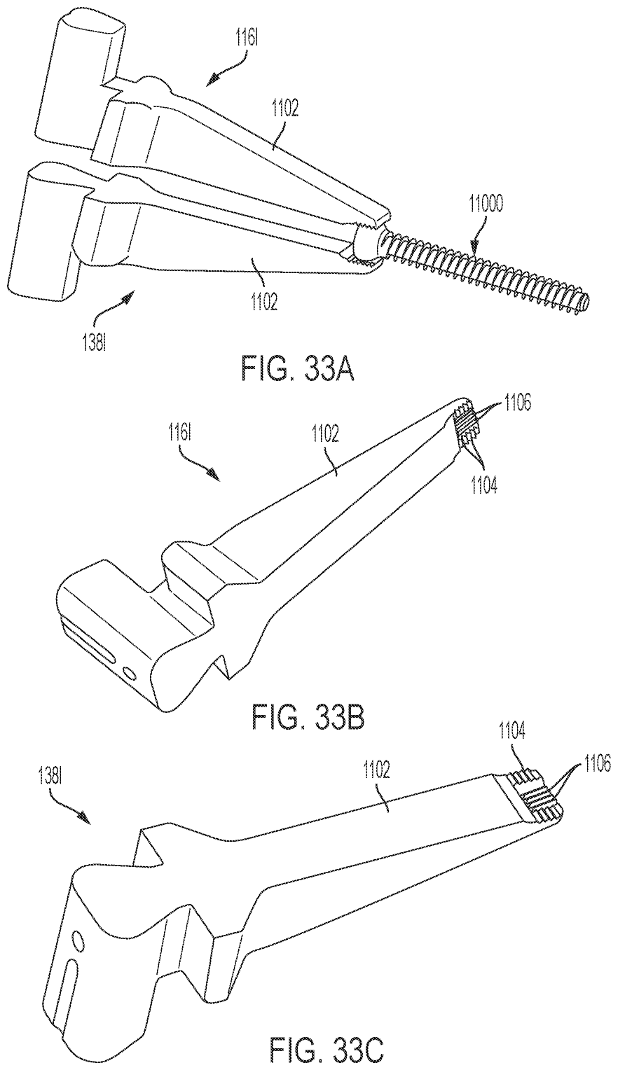

Referring to FIGS. 33A-33C there is shown a pair of jaws 116I, 138I which are attachable to the first arm 108 and link 130, respectively (FIG. 1). Jaws 116I, 138I are suitable for gripping an end, e.g., head, of an object 11000 such as a screw and each include a bite portion 1102. Distal ends of opposing gripping faces of the bite portions are provided with a plurality of transversely extending teeth or ridges 1104 and a plurality of longitudinally extending teeth or ridges 1106 for firmly gripping the end of the object.

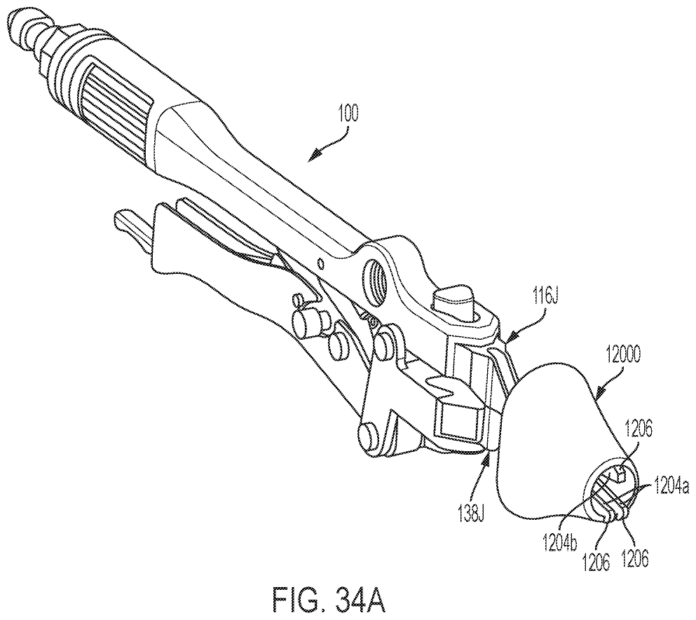

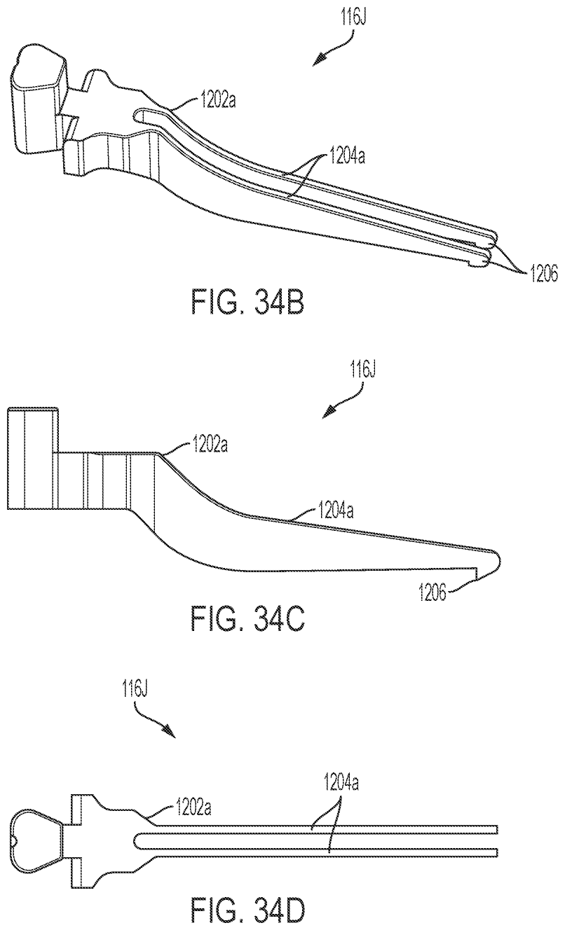

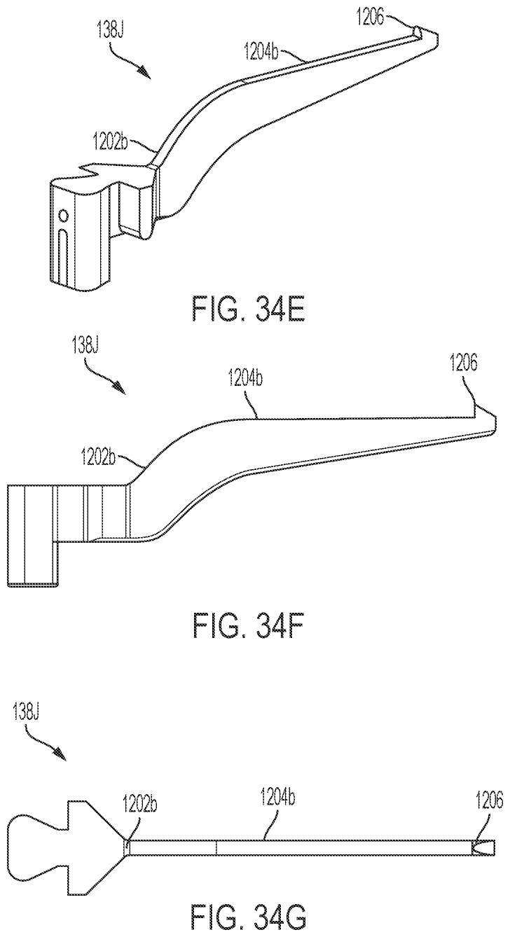

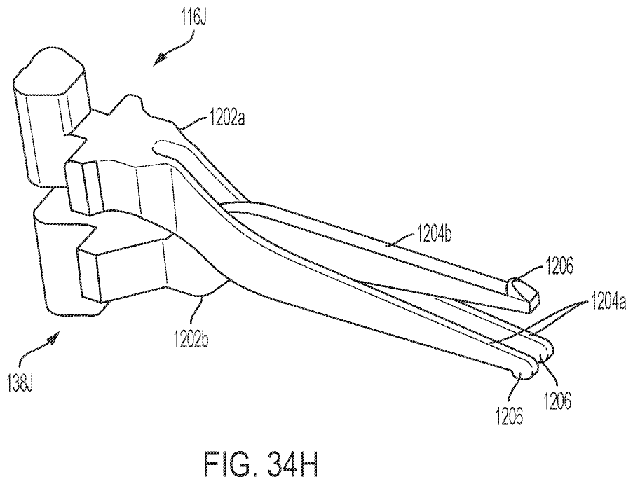

Referring to FIG. 34A, there is shown the surgical extractor 100 equipped with a pair of jaws 116J, 138J which are attachable to the first arm 108 and link 130, respectively (FIG. 1). Jaws 116J, 138J are suitable for engaging a distal end of an object 12000 such as a revision sleeve. As shown in FIGS. 34B-34D, the jaw 116J includes a bite portion 1202a comprising a pair of elongated bifurcated arms 1204a each having a downwardly depending lug 1206 at a distal end thereof. As shown in FIGS. 34E-34G, the jaw 138J includes a bite portion 1202b comprising a single elongated arm 1204b having an upwardly projecting lug 1206 at a distal end thereof. As shown in FIG. 34H, in operation, the single elongated arm 1204b of the jaw 138J passes through a gap defined between the pair of elongated bifurcated arms 1204a of the jaw 116J. As such, the lugs 1206 of the arms 1204a and arm 1024b engage a perimeter of an opening defined at the distal end of the object 12000 (FIG. 34A) whereby the object can be extracted from a patient's body by the surgical extractor 100.

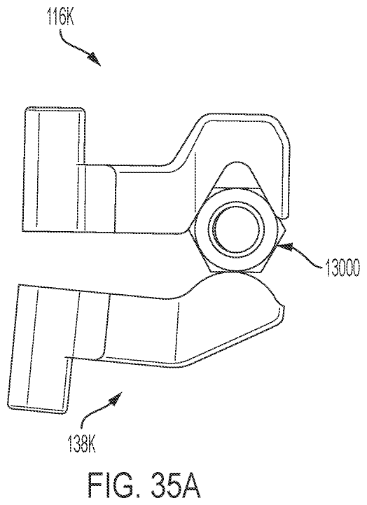

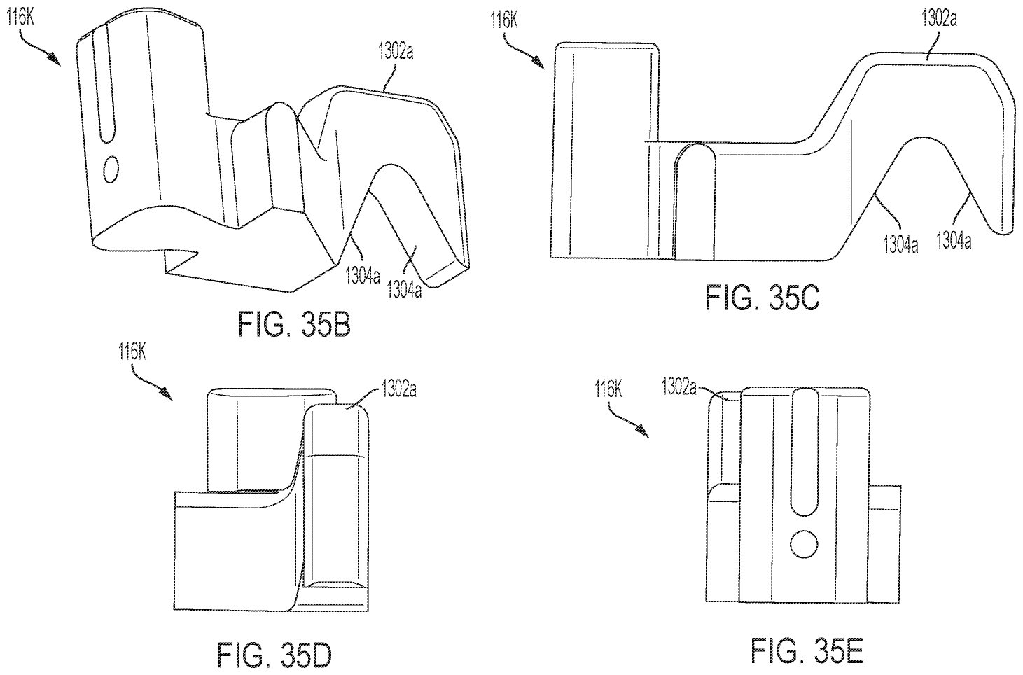

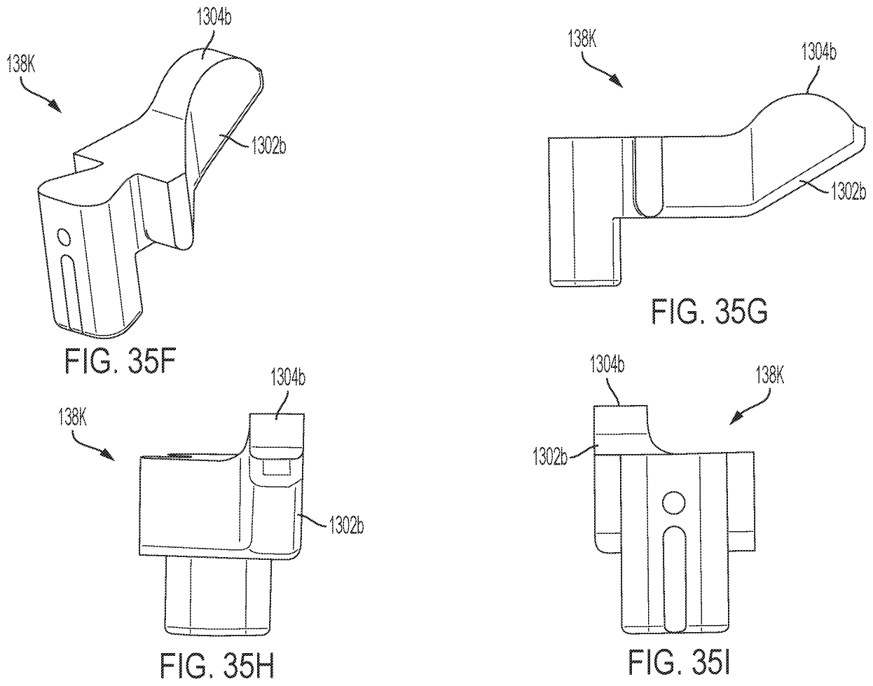

Referring to FIG. 35A, there is shown a pair of jaws 116K, 138K which are attachable to the first arm 108 and link 130, respectively (FIG. 1). Jaws 116K, 138K are suitable for engaging an object 13000 such as a hex nut. As shown in FIGS. 35B and 35C, the jaw 116K includes a bite portion 1302a comprising a pair of sloped surfaces 1304a defining an acute angle whereby the sloped surfaces substantially matingly engage first and second spaced apart surfaces of the hex nut (FIG. 35A). As shown in FIGS. 35D and 35E, the bite portion 1302a is laterally offset from the longitudinal center of the jaw 116K. As shown in FIGS. 35F and 35G, the jaw 138K includes a bite portion 1302b having an upwardly convex surface 1304b for engaging a third surface of the hex nut that is spaced apart from the surfaces of the hex nut engaged by the pair of sloped surfaces 1304a of the jaw 116K. As shown in FIGS. 35H and 35I, the bite portion 1302b is laterally offset from the longitudinal center of the jaw 138K a distance substantially equal to the lateral offset of the bite portion 1302a of the jaw 116K such that the bite portions 1302a, 1302b are in substantial lateral alignment in operation. The lateral offsets of the bite portions 1302a, 1302b are provided in order to enable lateral surfaces of the bite portions to abut against bone or other bodily tissue whereby essentially the entirety of the bite portions engage the object 13000 for optimum gripping thereof.

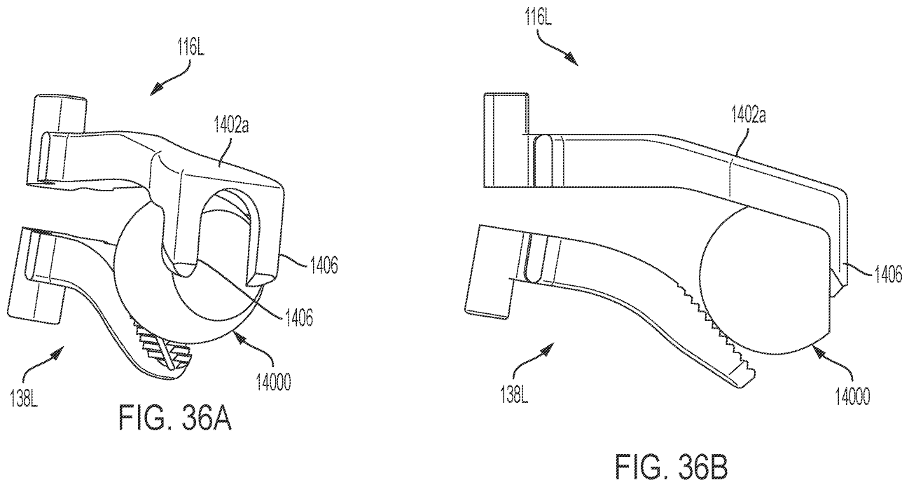

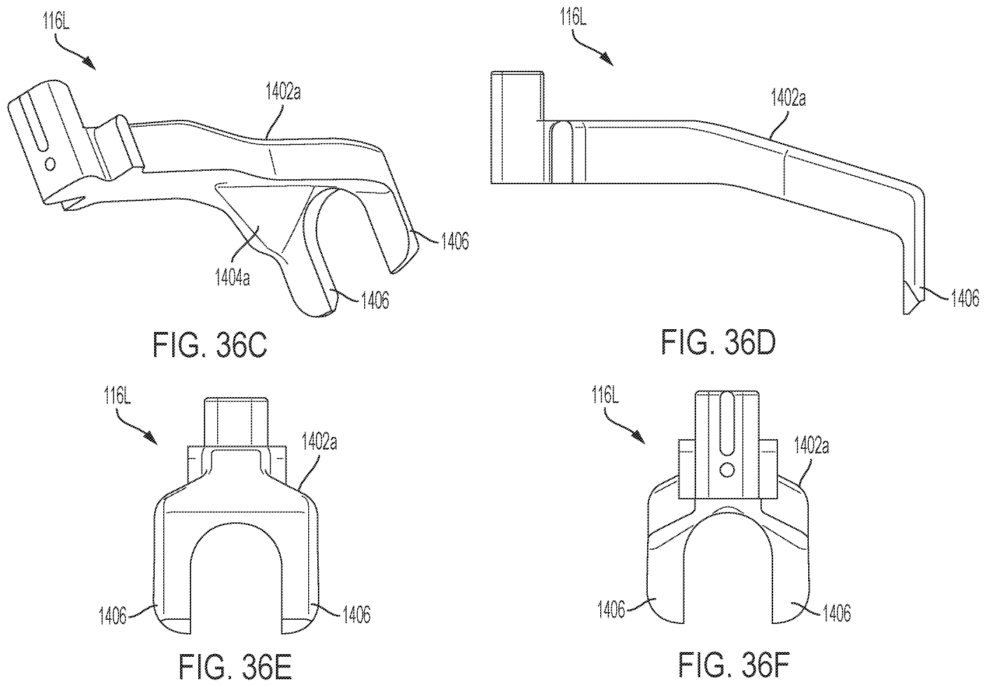

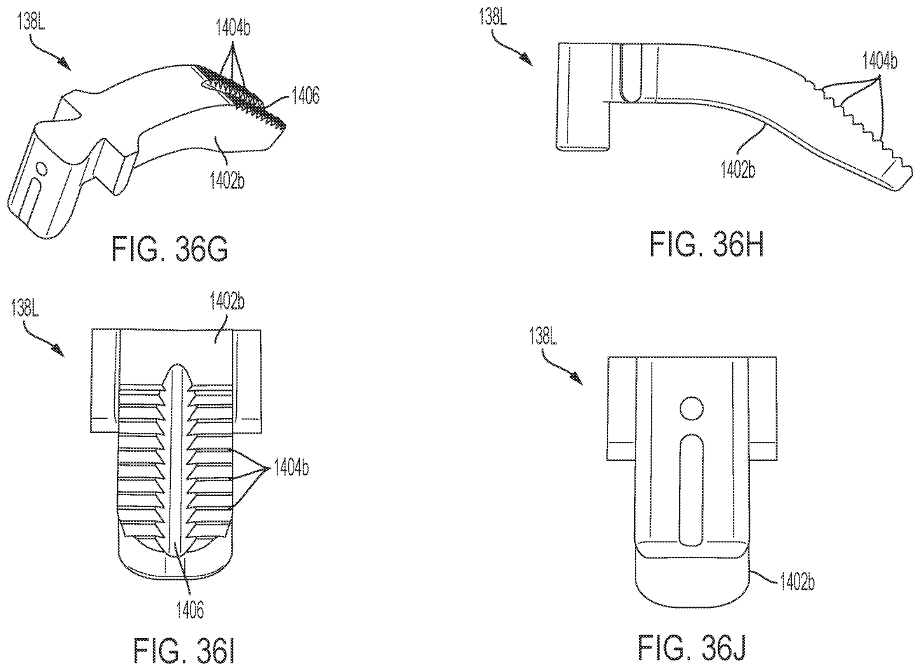

Referring to FIGS. 36A and 36B, there is shown a pair of jaws 116L, 138L which are attachable to the first arm 108 and link 130, respectively (FIG. 1). Jaws 116L, 138L are suitable for engaging an object 14000 such as a femoral head implant. The jaw 116L includes a bite portion 1402a including a generally triangular-shaped recess 1404a (FIG. 36C) configured to receive and grip a substantially spherical outer surface of the femoral head implant. The distal end of the bite portion 1402a is provided with a pair of downwardly depending arms 1406 (FIGS. 36C-36F) which are configured to engage a distal face of the femoral head implant, as shown in FIGS. 36A and 36B. As shown in FIGS. 32G-32J, the jaw 138L includes a bite portion 1402b that curves downwardly from a proximal end to a distal end thereof. A gripping face of the bite portion 1402b is provided with a plurality of transversely extending teeth or ridges 1404b for firmly gripping the femoral head implant. To further enhance gripping of the implant, the jaw 138L can include a substantially centrally disposed longitudinal groove 1406. Provision of such a groove increases the number of teeth edges which engage the substantially spherical outer surface of the femoral head implant.

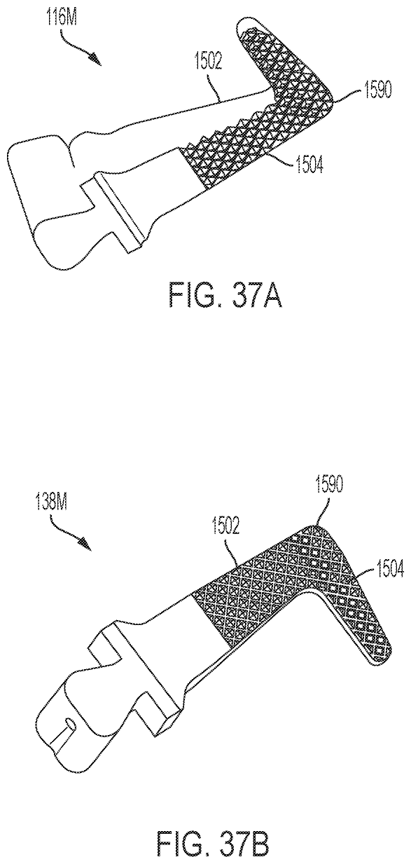

Referring to FIGS. 37A and 37B, there is shown a pair of jaws 116M, 138M which are attachable to the first arm 108 and link 130, respectively (FIG. 1). Each of the jaws 116M and 138M includes a bite portion 1502 which tapers from a proximal end to a distal end thereof and includes a bend 1590 about a mid-portion thereof. Bend 1590 is illustrated as being substantially 90 degrees, although it will be understood that the bend may be greater or less than 90 degrees. Opposed gripping faces of the bite portion of the jaws 116M, 138M may be provided with ridge-like teeth such as those shown in other jaw embodiments described hereabove. Alternatively, as illustrated, the opposed gripping faces may be provided with knurling or similarly toothed gripping structure 1504.

It will be appreciated by those skilled in the art that changes could be made to the exemplary embodiments described above without departing from the broad inventive concept thereof. It is to be understood, therefore, that this disclosure is not limited to the particular exemplary embodiments disclosed, but it is intended to cover modifications within the spirit and scope of the subject disclosure and as defined by the appended claims.

I claim:

1. A surgical extractor comprising:
a second arm having a proximal end and a distal end;
a first arm having a proximal end for attachment to an extraction device and a distal end for attachment to a first jaw;
a link pivotably connected to the first and second arms, the link having a distal end for attachment to a second jaw; and
a first jaw releasably attachable to the distal end of the first arm and a second jaw releasably attachable to the distal end of the link.

2. The surgical extractor of claim 1, wherein the first and second jaws each include a slidable lock to slidingly engage a corresponding slidable lock on the first arm and link, respectively.

3. The surgical extractor of claim 2, wherein the corresponding slidable lock on the first arm and link each includes a stop.

4. The surgical extractor of claim 2, wherein the slidable lock is a dovetail.

5. The surgical extractor of claim 2, wherein the slidable lock on each of the first and second jaws is a male dovetail and the corresponding slidable lock on each of the first arm and the link is a female dovetail.

6. The surgical extractor of claim 2, further comprising a detent carried by one of the first jaw and the first arm or a detent carried by one of the second jaw and the link.

7. The surgical extractor of claim 1, wherein a gripping face of a bite portion of at least one of the first jaw and the second jaw comprises a plurality of transverse teeth and a longitudinal groove separating the plurality of transverse teeth, a projection which stands proud of the gripping face and is configured to engage a slot of an object, a lip-like projection which stands proud of the gripping face and is configured to engage a distal face of an object, a plurality of transverse teeth arranged in a concave arc, or a plurality of transversely extending teeth and a plurality of longitudinally extending teeth.

8. The surgical extractor of claim 1, wherein a bite portion of the first jaw comprises a pair of elongated bifurcated arms each having a downwardly depending lug at a distal end thereof, wherein a bite portion of the second jaw comprises a single elongated arm having an upwardly projecting lug at a distal end thereof, and wherein the single elongated arm of the second jaw is operable to pass through a gap defined between the pair of elongated bifurcated arms of the first jaw, whereby the lugs of the elongated bifurcated arms and the lug of the single elongated arm engage a perimeter of an opening of an object.

9. The surgical extractor of claim 1, wherein a bite portion of the first jaw comprises a pair of sloped surfaces defining an acute angle for engaging first and second spaced apart surfaces of a hex nut, and wherein a bite portion of the second jaw has a convex surface for engaging a third surface of the hex nut that is spaced apart from the first and second spaced apart surfaces of the hex nut.

10. The surgical extractor of claim 1, wherein a bite portion of the first jaw comprises a pair of downwardly depending arms at a distal end thereof, and wherein a bite portion of the second jaw curves downwardly from a proximal end to a distal end thereof.

11. A surgical extractor comprising:
a second arm having a proximal end and a distal end;
a first arm having:
a proximal end for attachment to an extraction device,
a distal end for attachment to a first jaw, and
a fastener configured to releasably retain an extraction impactor or an extraction device connector, and
a link pivotably connected to the first and second arms, the link having a distal end for attachment to a second jaw.

12. The surgical extractor of claim 11, further comprising an extraction impactor or an extraction device connector having a cooperating fastener for engaging the fastener of the first arm.

13. The surgical extractor of claim 12, wherein the fastener of the first arm and the cooperating fastener of the impact extractor or the extraction device connector are selected from the group comprising cooperating threading, a J-slot connection, a press-fit, a Luer lock, a snap fit, and a ball detent.

14. The surgical extractor of claim 12, wherein the extraction impactor comprises a radially projecting knob defining a striking surface.

15. The surgical extractor of claim 14, wherein the radially projecting knob comprises a plurality of grip enhancing notches provided on a periphery thereof.

16. The surgical extractor of claim 12, wherein the extraction device connector comprises a base for receiving a strike plate.

17. The surgical extractor of claim 16, wherein the base is polygonal in shape.

18. The surgical extractor of claim 12, wherein the extraction device connector comprises a quick connect configured to releasably engage with an extraction device.

19. The surgical extractor of claim 12, wherein the extraction impactor or the extraction device connector extends laterally away from the first arm when connected to the fastener.

20. The surgical extractor of claim 12, wherein the fastener is an opening transverse to a longitudinal axis of the first arm.

21. The surgical extractor of claim 12, wherein the fastener is positioned spaced from the distal end of the first arm.

22. The surgical extractor of claim 12, wherein the fastener is positioned about a midportion of the first arm.

23. The surgical extractor of claim 12, wherein the fastener is between the distal end of the first arm for attaching to the first jaw and a proximal end of the first arm.

24. A surgical extractor kit comprising:
a surgical extractor that includes:
a second arm having a proximal end and a distal end,
a first arm having a proximal end for attachment to an extraction device and a distal end for attachment to a first jaw, and
a link pivotably connected to the first and second arms, the link having a distal end for attachment to a second jaw;
a first pair of jaws including a first jaw releasably attachable to the distal end of the first arm and a second jaw releasably attachable to the distal end of the link, wherein a gripping face of a bite portion of at least one of the first jaw and the second jaw comprises a plurality of transverse teeth and a longitudinal groove separating the plurality of transverse teeth, a projection which stands proud of the gripping face and is configured to engage a slot of an object, a lip-like projection which stands proud of the gripping face and is configured to engage a distal face of an object, a plurality of transverse teeth arranged in a concave arc, or a plurality of transversely extending teeth and a plurality of longitudinally extending teeth;
a second pair of jaws including a first jaw releasably attachable to the distal end of the first arm and a second jaw releasably attachable to the distal end of the link, wherein a bite portion of the first jaw comprises a pair of elongated bifurcated arms each having a downwardly depending lug at a distal end thereof, wherein a bite portion of the second jaw comprises a single elongated arm having an upwardly projecting lug at a distal end thereof, and wherein the single elongated arm of the second jaw is operable to pass through a gap defined between the pair of elongated bifurcated arms of the first jaw, whereby the lugs of the elongated bifurcated arms and the lug of the single elongated arm engage a perimeter of an opening of an object;
a third pair of jaws including a first jaw releasably attachable to the distal end of the first arm and a second jaw releasably attachable to the distal end of the link, wherein a bite portion of the first jaw comprises a pair of sloped surfaces defining an acute angle for engaging first and second spaced apart surfaces of a hex nut, and wherein a bite portion of the second jaw has a convex surface for engaging a third surface of the hex nut that is spaced apart from the first and second spaced apart surfaces of the hex nut; and
a fourth pair of jaws including a first jaw releasably attachable to the distal end of the first arm and a second jaw releasably attachable to the distal end of the link, wherein a bite portion of the first jaw comprises a pair of downwardly depending arms at a distal end thereof, and wherein a bite portion of the second jaw curves downwardly from a proximal end to a distal end thereof.
